(19) United States
(12) Patent Application Publication
(10) Pub. No.: US 2020/0121206 A1
(43) Pub. Date: Apr. 23, 2020

(54) ELECTROENCEPHALOGRAPHY DEVICE AND DEVICE FOR MONITORING A SUBJECT USING NEAR INFRARED SPECTROSCOPY

(71) Applicant: The University of British Columbia, Vancouver (CA)

(72) Inventors: Guy Dumont, Vancouver (CA); Shahbaz Askari, Vancouver (CA); Zoya Jafaryrabanybastany, Vancouver (CA)

(21) Appl. No.: 16/625,672

(22) PCT Filed: Jun. 26, 2018
(86) PCT No.: PCT/CA2018/050787

§ 371 Date: Dec. 20, 2019

Related U.S. Application Data
(60) Provisional application No. 62/524,679, filed on Jun. 26, 2017.
(60) Provisional application No. 62/524,683, filed on Jun. 26, 2017.

(51) Int. Cl. A61B 5/04; A61B 5/0478

(52) U.S. Cl. CPC A61B 5/04004; A61B 5/0478

(57) ABSTRACT

There is described are electroencephalography (EEG) device. The EEG device comprises one or more inputs for connecting via electrodes to a subject, and one or more DC/AC EEG amplifiers for amplifying electric potential signals from a subject. Each DC/AC EEG amplifier comprises a preamplifier connected to the one or more inputs and configured to amplify a signal received at the one or more inputs, the preamplifier having a differential gain set so as to have a common mode rejection ratio (CMRR) of at least 100 dB. Each DC/AC EEG amplifier further comprises an amplifier circuit connected to the preamplifier and configured to further amplify the signal, and, based thereon, output an output signal comprising a DC component having a frequency of less than 0.02 Hz. There is also described a device for monitoring a subject using near-infrared spectroscopy (NIRS) and electroencephalography (EEG), comprising: one or more light emitters each configured to emit toward the subject light having a wavelength in a range of 650 nm to 1,100 nm; one or more modulators configured to modulate the emitted light using one or more carrier frequencies; one or more light detectors configured to detect modulated light reflected from the subject one or more demodulators configured to demodulate the detected light, based on the one or more carrier frequencies; and one or more EEG electrodes on to measure electric potentials of the subject.

### CROSS-REFERENCE TO RELATED APPLICATIONS

This application is a US national phase of International Patent Application No. PCT/CA18/50787 filed Jun. 26, 2018; which claims priority from US Provisional Patent Application Nos. 62/524,679 filed Jun. 26, 2017 and 62/524,683 filed Jun. 26, 2017, which applications are incorporated herein by reference in their entireties.

### TECHNICAL FIELD

The present disclosure relates to an electroencephalography (EEG) device and to a method of using the EEG device. The disclosure further relates to a device for monitoring a patient using near infrared spectroscopy (NIRS) and EEG, as well as to a method of monitoring a patient using NIRS.

### BACKGROUND

Electroencephalography (EEG) measurements are based on the diffusion of potassium ions (K⁺) within brain cells and sodium ions (Na⁺) outside of neurons. These transmissions create ionic currents along the cell membranes in intra- and extracellular spaces. The portion of the generated current in extracellular spaces produces two types of field potentials, distinguishable when recording the field potentials against an inactive reference point. If a recording device captures the field potentials with a time constant less than one second, a first type of alternating current EEG (AC EEG) is recognizable. A second type known as the direct current potential (DC potential—also referred to as ultra-low frequency EEG) is recognizable for much longer time constants. The first type of field potential (AC EEG) is usually described in terms of brain electrical activity in different frequency bands, such as delta (δ-band: 0.5-4 Hz), theta (θ-band: 4-7 Hz), alpha (α-band: 8-12 Hz), beta (β-band: 12-30 Hz), and gamma (γ-band: 30-100 Hz). The second type of field potential (DC EEG) is generally defined in terms of brain electrical activity having frequencies less than 0.1 Hz. Recent studies indicate the existence of the near-DC field potential (the second type) with a duration that can reach up to 120 s; therefore, the EEG frequency range in the lower band needs to extended to 0.01 Hz.

The field potential amplitude recorded by scalp electrodes typically varies in a range of 5 pV to 200 pV for a normal AC EEG, and rises to 400 pV for spike waves associated with epilepsy. Furthermore, the amplitudes of the spike waves can reach up to 1 mV when recorded through electrocorticography (ECoG) electrodes. Still further, when recorded using invasive techniques, DC EEGs have been shown to reach amplitudes of 30 mV. However, there currently is not believed to exist a method for accurately estimating DC potential amplitude ranges using non-invasive techniques, e.g. from the surface of the scalp.

Spreading depression (SD) is a negative DC potential and remains one of the most significant EEG patterns in DC EEG. SD is a major transient, localized relocation of ions between the extracellular and intracellular spaces that causes a neuronal excitation accompanied by depolarization and a period of electrical silence. SD plays a crucial role in abnormal brain activities such as a migraine, cerebrovascular diseases, and epilepsy. SD deflection propagates at 3-5 mm/min in the neuronal tissues with an amplitude of 2-30 mV and a duration of 30-120 s. It has the longest duration among known DC EEG patterns. Currently, this valuable data has only been recorded using ECoG electrodes, due to technical barriers posed by the recording of SD waves from the scalp surface. Although the recording of DC cortical field potentials provides additional information on brain electrical activities, the invasiveness of ECoG is severely limiting to its widespread use.

The asymmetric potential changes of the electrode-gel-skin interface at ultralow frequencies, an effect of the cortex-scalp distance, also pose technical challenges for non-invasive recording of SD. For non-invasive DC EEG measurements, the amplification of a large amplitude range of 5 pV to 30 mV creates other challenges including the saturation of the amplifier resulting from electrode and tissue half-cell potentials.

Another method of non-invasive monitoring of brain activity is near-infrared spectroscopy (NIRS). NIRS can be used as a non-invasive neuroimaging method to detect hemodynamic changes in the cerebral cortex. NIRS takes advantage of the fact that skin, bone and other tissues of the human body are almost transparent in the near-infrared spectrum (˜700-900 nm, also known as the “optical window”), whereas oxyhemoglobin (HbO₂) and deoxyhemoglobin (HHb) are highly sensitive to infrared light. In the spectrum of the optical window, infrared light can sample the biological tissues with appropriate depth of penetration. Water and hemoglobin components of the tissue massively absorb infrared radiation with wavelengths greater than 900 nm. Therefore, the optimum spectral range for cerebral spectroscopy is the “NIR optical window”, i.e. the spectrum in which light is largely absorbed by HbO₂ and HHb. In the NIR optical window, other substances, such as collagen and fat, are sensitive to NIR radiation and may absorb near-infrared wavelengths more strongly than HbO₂ and HHb. However, the high absorption coefficients of these biological materials are generally not a major issue in cerebral NIR since the concentrations of these materials are dramatically lower relative to the concentration of HbO₂ and HHb in cerebral N IRS.

In this context, there is a need for improved devices and techniques for performing EEG, particularly in the ultra-low frequency range (DC EGG), as well as for performing NIRS.

### SUMMARY

In a first aspect of the disclosure, there is provided an electroencephalography (EEG) device comprising: one or more inputs for connecting via electrodes to a subject; one or more DC/AC EEG amplifiers for amplifying electric potential signals from a subject, wherein each DC/AC EEG amplifier comprises: a preamplifier connected to the one or more inputs and configured to amplify a signal received at the one or more inputs, the preamplifier having a differential gain set so as to have a common mode rejection ratio (CMRR) of at least 100 dB; and an amplifier circuit connected to the preamplifier and configured to further amplify the signal and, based thereon, output an output signal comprising a DC component having a frequency of less than 0.02 Hz.

The one or more inputs may comprise multiple inputs including at least one reference input and one or more EEG inputs. The EEG device may further comprise a reference circuit connected to the at least one reference input and configured to output a reference signal based on a signal received at the at least one reference input. The preamplifier may be further connected to the reference circuit and may be further configured to amplify the signal received at the one or more EEG inputs. The reference signal may be buffered prior to being received at the preamplifier. The reference circuit may comprise a multi-turn potentiometer for compensating resistor tolerance. The reference circuit may comprise a transient voltage suppression (TVS) diode, and each preamplifier may comprise a TVS diode connected at an input thereto.

The EEG device may further comprise one or more recorders configured to record the output signal.

The CMRR may be at least 100 dB at 50 Hz or 60 Hz. In particular, the CMRR may be at least 110 dB at 50 Hz or 60 Hz.

The DC component may have a frequency of from 0.015 Hz to 0.02 Hz. In some cases, for example when neither the pre-amplifier nor the amplifier circuit comprises any high-pass filter, the DC component may have a frequency less than 0.015 Hz.

The output signal may further comprise an AC component having a frequency of from 0.02 Hz to 80 Hz. The one or more recorders may be further configured to simultaneously record the DC component and the AC component.

The one or more recorders comprise one or more of: circuitry; and one or more processors communicative with a computer-readable medium comprising computer program code stored thereon and configured when read by the one or more processors to cause the one or more processors to record the output signal.

The EEG device may further comprise one or more electrodes for connecting to the one or more inputs and for attaching to a subject's scalp. The electrodes may comprise silver-chloride electrodes. The electrodes may be non-polarizable.

The differential gain may be set to about 20, and in particular may be set to 19.93. A combined differential gain of the pre-amplifier and the amplifier circuit may be set to about 200.

Each preamplifier may comprise an operational amplifier having an input impedance greater than 10 GO.

Each preamplifier may comprise an operational amplifier having an input bias current of less than 10 pA.

The EEG device may further comprise a Driven Right Leg (DRL) circuit for connecting via one or more electrodes to a subject, and for reducing a common mode voltage.

The differential gain may be set by a single resistive component.

In some embodiments, each preamplifier does not comprise a high-pass filter.

A first multilayer PCB may comprise each preamplifier in a topmost layer of the first PCB, and one or more inputs in a bottommost layer of the first PCB. A second PCB may comprise each amplifier circuit.

The DC component may have a frequency indicative of cortical spreading depression.

In some embodiments, each amplifier circuit: does not comprise a high-pass filter;

or comprises an integrator.

In a further aspect of the disclosure, there is provided a method of recording electroencephalography (EEG) signals from a subject, comprising: attaching one or more electrodes to the subject's scalp, the electrodes connected to one or more inputs of an EEG device, the EEG device comprising one or more DC/AC EEG amplifiers for amplifying electric potential signals from the subject, wherein each DC/AC EEG amplifier comprises: a preamplifier connected to the one or more inputs and configured to amplify a signal received at the one or more inputs, the preamplifier having a differential gain set so as to have a common mode rejection ratio (CMRR) of at least 100 dB; and an amplifier circuit connected to the preamplifier and configured to further amplify the signal and, based thereon, output an output signal comprising a DC component having a frequency of less than 0.02 Hz; and using the EEG device to record electric potentials of the subject.

The subject may be epileptic.

The EEG device may comprise any of the features described above in connection with the first aspect of the disclosure.

In a further aspect of the disclosure, there is provided a device for monitoring a subject using near-infrared spectroscopy (NIRS) and electroencephalography (EEG), comprising: one or more light emitters each configured to emit toward the subject light having a wavelength in a range of 650 nm to 1,100 nm; one or more modulators configured to modulate the emitted light using one or more carrier frequencies; one or more light detectors configured to detect modulated light reflected from the subject; one or more demodulators configured to demodulate the detected light, based on the one or more carrier frequencies; and one or more EEG electrodes configured to measure electric potentials of the subject. The method may be used to monitor changes in local cerebral blood flow and local cerebral oxygenation in a subject, and may be useful in the study of epilepsy, cerebral activation and the identification of hypoxic ischemia injury.

In comparison to other brain imaging techniques (e.g. functional magnetic resonance imaging [fMRI] and positron emission tomography [PET]), NIRS has advantages such as its portability and low cost. In addition, NIRS uses non-ionizing electromagnetic radiation in the near-infrared spectrum, a safe form of radiation; NIRS measurements are not harmful and have been categorized as a non-invasive imaging method. Furthermore, challenges created by motion artifacts due to the subject's slight movements can be overcome, whereas motion artifacts are still a serious issue in the aforementioned imaging modalities. NIRS can be used to measure hemodynamic changes in small capillaries, while fMRI can only accomplish measurements in small vessels. Therefore, NIRS measurements are more accurate for monitoring the local hemodynamic characteristics of the tissue on the microscopic scale. Combining NIRS and EEG signals may provide useful concurrent information about neuronal activity and blood flow to tissue.

The one or more light detectors may comprise one or more optodes.

Each EEG electrode may be positioned between one of the light emitters and one of the light detectors.

The one or more light detectors may comprise multiple light detectors arranged along two or more rows, and the one or more light emitters may comprise multiple light emitters arranged between the two or more rows of light detectors.

The one or more carrier frequencies may comprise one or more of 1.5 kHz, 15 kHz, 54 kHz, and 85 kHz.

The emitted light may comprise wavelengths of one or more of 740 nm, 780 nm, 850 nm, and 950 nm.

The one or more modulators may be further configured to modulate the emitted light using continuous wave amplitude modulation.

Adjacent light detectors may be separated by about 28 mm, and adjacent EEG electrodes may be separated by about 12 mm.

The one or more: light emitters, light detectors, and EEG electrodes may be attached to a flexible support for positioning on a forehead of a subject.

The device may further comprise one or more bandpass filters each comprising one or more circuit components, the one or more bandpass filters configured to filter the detected light according to one or more specific wavelengths.

Each light source may be positioned at a centre of a square defined by locations of four of the one or more light detectors.

Each light source may be positioned at a centre of a square defined by locations of four of the one or more EEG electrodes.

The electric potentials may comprise electric potentials of a brain of a subject.

Each EEG electrode may be connected to a DC/AC EEG amplifier comprising: a preamplifier configured to amplify a signal received via the EEG electrode, the preamplifier having a differential gain set so as to have a common mode rejection ratio (CMRR) of at least 100 dB; and an amplifier circuit connected to the preamplifier and configured to further amplify the signal and, based thereon, output an output signal comprising a DC component having a frequency of less than 0.02 Hz.

Each DC/AC EEG amplifier may comprise any of the features described above in connection with the first aspect of the disclosure.

In a further aspect of the disclosure, there is provided a method of monitoring a subject using near-infrared spectroscopy (NIRS), comprising: emitting toward the subject light having a wavelength in a range of 650 nm to 1,100 nm; modulating the emitted light using one or more carrier frequencies; detecting the modulated light reflected from the subject; and demodulating, based on the one or more carrier frequencies, the detected light.

The method may further comprise recording an amplitude of the demodulated light.

The method may further comprise recording electric potentials of the subject. The electric potentials may be recording concurrently to the recording of the amplitude of the demodulated light. The electric potentials may be recorded using one or more electroencephalography (EEG) electrodes.

### BRIEF DESCRIPTION OF THE DRAWINGS

Detailed embodiments of the disclosure will now be described in conjunction with the accompanying drawings of which:

FIG. 1 is a circuit diagram of a DC/AC EEG amplifier in accordance with an embodiment of the disclosure;

FIG. 2 is a circuit diagram of a DC/AC EEG amplifier in accordance with another embodiment of the disclosure;

FIGS. 3A and 3B show a circuit diagram of a multi-channel DC/AC EEG device in accordance with an embodiment of the disclosure;

FIG. 4A shows a first PCB of an EEG device in accordance with an embodiment of the disclosure;

FIG. 4B shows a second PCB of an EEG device in accordance with an embodiment of the disclosure;

FIG. 5A shows the first PCB of the EEG device;

FIG. 5B shows the second PCB of the EEG device;

FIG. 5C shows the combined first and second PCBs;

FIG. 6: shows the completed EEG device;

FIG. 7A shows a recorded spreading depression (SD) wave from a rat scalp;

FIG. 7B is a power spectrogram of the signal of FIG. 7A;

FIG. 8 shows a an SD signal;

FIG. 9 shows an SD wave and a power spectrogram of the same;

FIG. 10 shows a three-dimensional window constructed from a Parzen window (n=m=40 points);

FIG. 11 shows an SD wave: “A” and “B” denote EEG signals after KCl injection; “C” denotes phase 1; “D” denotes phase 2; “E” is between phases 2 and 3; “F” denotes phase 3; “G” denotes the depression state; and “H” denotes the recovery state of the SD wave;

FIG. 12 shows two SD waves captured on a patient's left occipital lobe;

FIG. 13 shows a power spectrogram of the signal of FIG. 12 (the dark red indicates high energy and the dark blue indicates low energy);

FIG. 14 shows the propagation of an SD wave from P4 to O2 and then to PZ; the start of seizure activity is marked with the black line;

FIG. 15 shows power spectrograms of the SD wave of FIG. 14 (the dark red indicates high energy and the dark blue indicates low energy);

FIG. 16 shows seizure electrical activity after the SD wave of FIGS. 14 and 15;

FIG. 17 shows power spectrograms of the SD waves (the dark red indicates high energy and the dark blue indicates low energy); this figure shows that the amplifier can capture ultra-low frequency content of less than 0.02 Hz;

FIG. 18 shows a combined NIRS and EEG device, in accordance with an embodiment of the disclosure;

FIGS. 19A and 19B are schematics of the emission, modulation, detection, filtering, and demodulation of NIRS light;

FIG. 20 is a circuit of a Sallen-Key bandpass filter;

FIG. 21 shows a light path between an NIRS emitter and an NIRS detector, and an EEG electrode positioned between the emitter and the detector;

FIG. 22 is a spectrum of ambient light;

FIGS. 23A and 23B are frequency responses of four Sallen-Key bandpass filters;

FIG. 24 represents schematically a method of simulating contamination of an input signal;

FIG. 25 are plots illustrating amplitude modulation of an NIRS signal;

FIGS. 26A, 26B and 26C show a combined NIRS and EEG device, in accordance with an embodiment of the disclosure;

FIG. 27 is a block diagram of the NIRS and EEG device of FIGS. 26A, 26B and 26C;

FIG. 28 depicts the change in HbO₂ and HHb during arterial occlusion;

FIG. 29 shows the placement of EEG B1 and B2 electrodes on a subject;

FIG. 30 shows the hemodynamic response to a cold pressor test (CPT); the first diagram represents the measurement of optical density at the wavelengths 740 nm and 850 nm;

FIG. 31 shows neuronal activity and hemodynamic variation during the CPT;

FIG. 32 shows changes to Gamma-band power during the CPT;

FIG. 33 shows Gamma-band oscillations during the CPT (the green signal represents the total Hb during the experiment);

FIG. 34 shows a time-frequency transform of an EEG signal in the Gamma band (the green signal represents the total Hb during the experiment);

FIG. 35 shows measurements of EEG and hemodynamic changes (HHb, HbO₂, and total Hb) induced by hypoxic breathing; the upper diagram is the optical density of the tissue at 740 nm and 850 nm wavelengths;

FIG. 36 shows measurements of EEG in the frontal and sensory cortex during hypoxic breathing; the upper diagram is the optical density of the tissue at 740 nm and 850 nm wavelengths;

FIG. 37 shows time-frequency analysis of the EEG signal based on STFT (Short-Time Fourier Transform); and

FIG. 38 shows a superposition of the EEG signal A1 (shown in green) and HHb multiplied by 50K (shown in blue).

### DETAILED DESCRIPTION

The present disclosure seeks to provide improved devices and techniques for performing EEG and NIRS. While various embodiments of the disclosure are described below, the disclosure is not limited to these embodiments, and variations of these embodiments may well fall within the scope of the disclosure which is to be limited only by the appended claims.

### DC/AC EEG Device

According to the present disclosure, there is described an amplifier for use with an EEG device to non-invasively capture DC EEG data. Using EEG electrodes connected to a patient, the EEG device is configured to non-invasively monitor EEG signals, and more particularly DC potentials having frequencies less than 0.02 Hz. The amplifier is configured to allow for simultaneous recording of both ultra-low and high-frequency EEG data from the human scalp, by opening the EEG frequency band from 0.2-70 Hz to 0.015-70 Hz, thereby providing additional information for diagnosing brain abnormalities. The AC component of EEG appears after and before the DC component of EEG in a spreading depression wave, and the two components mix under certain conditions, such as in Tonic-Clonic epilepsy. Therefore, simultaneously recording both DC and AC components of brain electrical activities is advantageous. Consequently, the EEG device is configured to record both ultra-low and high-frequency EEG data in sufficient quality to distinguish artifacts from the waveforms.

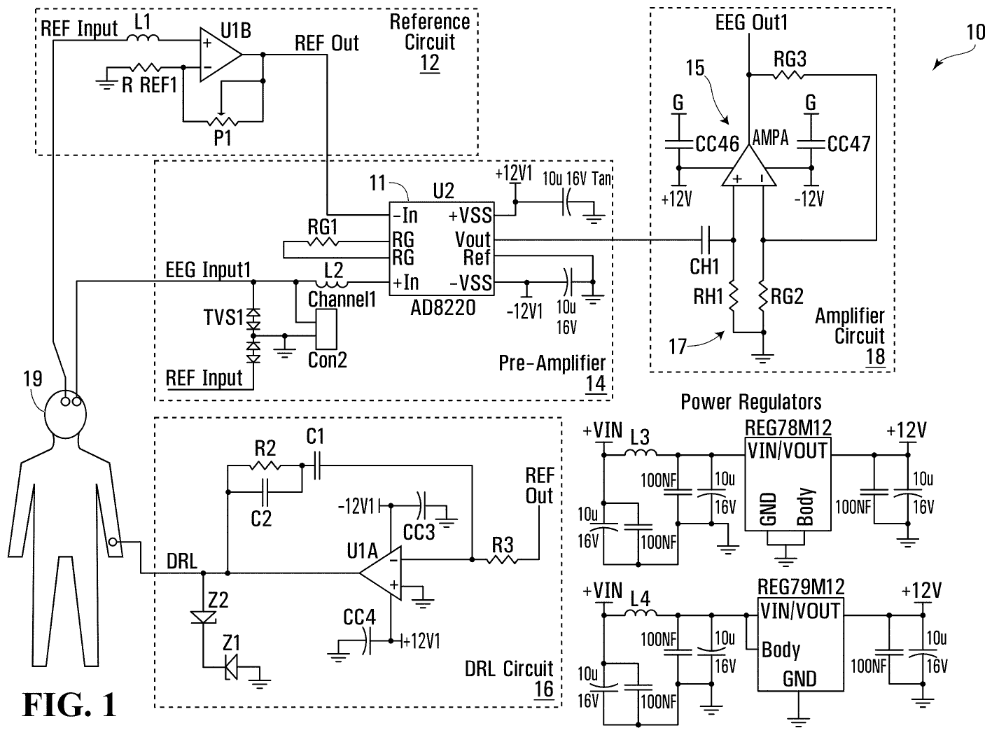

Turning to FIG. 1, there is shown an embodiment an EEG amplifier 10 in accordance with the present disclosure. EEG amplifier 10 comprises a reference circuit 12, a preamplifier 14, a DRL circuit 16, and an amplifier circuit 18. Preamplifier 14 comprises an operational amplifier (op-amp) 11, which in one implementation may be AD8220 (by Analog Devices, USA). AD8220 is a JFET input amplifier and provides a high input impedance above 10 GO. The bias current has a maximum of about 10 pA, with substantially no input bias current. Preamplifier 14 may operate from a dual power supply up to ±18 V and has rail-to-rail output capability for a higher dynamic range. This allows for the handling of the electrode-gel-skin interface's half-cell potential. Op-amp 11 may exceed a common mode rejection ratio (CMRR) of 110 dB with a differential gain set to 10, since its input impedance and capacitance are closely matched and placed near the input pins. Preamplifier 14 comprises laser-trimmed resistors for decreasing resistor tolerance. The above parameters may assist in keeping the CMRR in range of 86-140 dB.

In one embodiment, amplifier 10 comprises the following specifications:

| No | Factor | The value | |
|---|---|---|---|
| 1 | CMRR | >100 db at 50/60 Hz | |
| 2 | Overall ADIF | <200 | |
| 3 | Sample rate | 800 2K Hz | |
| 4 | Sensitivity | 5 μV-30 mV | |
| 5 | Input impedance | ≈10 | GΩ |
| 6 | Maximum input bias current | <nA | |
| 7 | DC saturation handling level | 800 | mV |
| 8 | Low frequency response min | ≈0.01 | Hz |
| | (−3 dB) | | |
| 9 | High-frequency response Max | >500 | Hz |
| | (3 dB) | | |
| 10 | Subject Isolation | =4 KV with low capacitance | |
| 11 | ESD protection | 4 KV by contact, 8 KV by air | |
| 12 | Channel number | Compatible with 10-0 and 10-20 | |
| | | EEG electrode placement systems | |
| 13 | Reference channels | >1 channel | |
| 14 | Samll size and low power | | |
| | consumption | | |

The patient 19 is driven by a buffered reference signal (REF OUT) through DRL circuit 16 to decrease the common mode potential, consequently increasing the CMRR. The reference signal (obtained from the patient's ears or nasion) is a good approximation of the average of the signal source; therefore, REF OUT is connected to the input of DRL circuit 16 (the inverting pin of U1A) and this signal is driven to the patient through DRL circuit 16's output (DRL). There is no direct connection between patient 19 and amplifier 10's power source, reducing the amplifier noise and improving patient safety.

Brain field potentials are recorded against an inactive reference point (REF INPUT)—usually at the patient's ears or nasion. Therefore, the reference signal captured by the EEG electrode from above the ear/nasion is buffered (REF OUT) and connected to the common or inverting input of op-amp 11. All EEG signals are compared against the reference signal, and amplifier 10 acts as a bipolar amplifier. The reference signal is buffered to avoid signal degradation loaded by sixty input channels.

In one particular implementation of amplifier 10, the differential gain of preamplifier 14 is set to 19.93 by using RG1=2.61 kΩ. This differential gain ensures a CMRR of at least 110 dB. The differential gain is set by using only one resistor, and therefore the CMRR in preamplifier 14 is largely insensitive to resistor tolerance.

Reference circuit 12 includes a multi-turn potentiometer P1 to compensate for resistor tolerance and to keep the overall CMRR above 110 dB. The common mode gain of preamplifier 14 becomes zero when the relevant impedances are selected according to:

(R REF1)/P1=1+(49.4 kΩ)/RG1 where ADIFF=1+(49.4 kΩ)/RG1

In addition, with the maximum half-cell potential of the electrode-gel-skin interface being about 790 mV peak-to-peak, preamplifier 14 is not saturated by setting the differential gain in preamplifier 14 to 19.93 and by supplying preamplifier 14 with a dual source voltage of ±12 V.

After bio-amplification of the EEG signal using preamplifier 14, the signal has adequate amplitude to be amplified by amplifier circuit 18. To avoid amplifier saturation due to the electrode half-cell potential, a high-pass filter 17 with a maximum cut-off frequency of 0.015 Hz is placed before amplifier circuit 18, and its gain is set to 10. In one particular implementation, amplifier circuit 18 comprises an op-amp 15 which may be TLV274, a rail-rail op-amp with low power consumption, high CMRR (approximately 110 dB), and a low input bias current.

The power source for amplifier 10 is an isolated ±15 V DC (4 kV with low capacitance). ±12 V voltage regulators with a pi filter are used for the power source to reduce the noise of ambient and power noise.

Two transient voltage suppression (TVS) diodes are included in the input of preamplifier 14 and in reference circuit 12 to comply with 4 kV ESD protection. Moreover, diodes Z1 and Z2 are included in DRL circuit 16 for further ESD protection.

The dimensions of EEG amplifier 10 may be reduced by using a single op-amp 11 for preamplifier 14, a single op-amp 15 for amplifier circuit 18, and a single resistance RG1 for setting the differential gain. In addition, op-amp 11 may be provided in an MSOP (Micro Small Outline Package) and op-amp 15 may be provided in a TSOP (Thin Small Outline Package), to further reduce the dimensions of EEG amplifier 10. Op-amp 11 may consume 750 pA and op-amp 15 may draw 550 pA of quiescent current, and the maximum power consumption for each channel of EEG amplifier 10 may be approximately 1.3 mA. Therefore, EEG amplifier 10 may have an overall power consumption of about 190 mA for sixty channels.

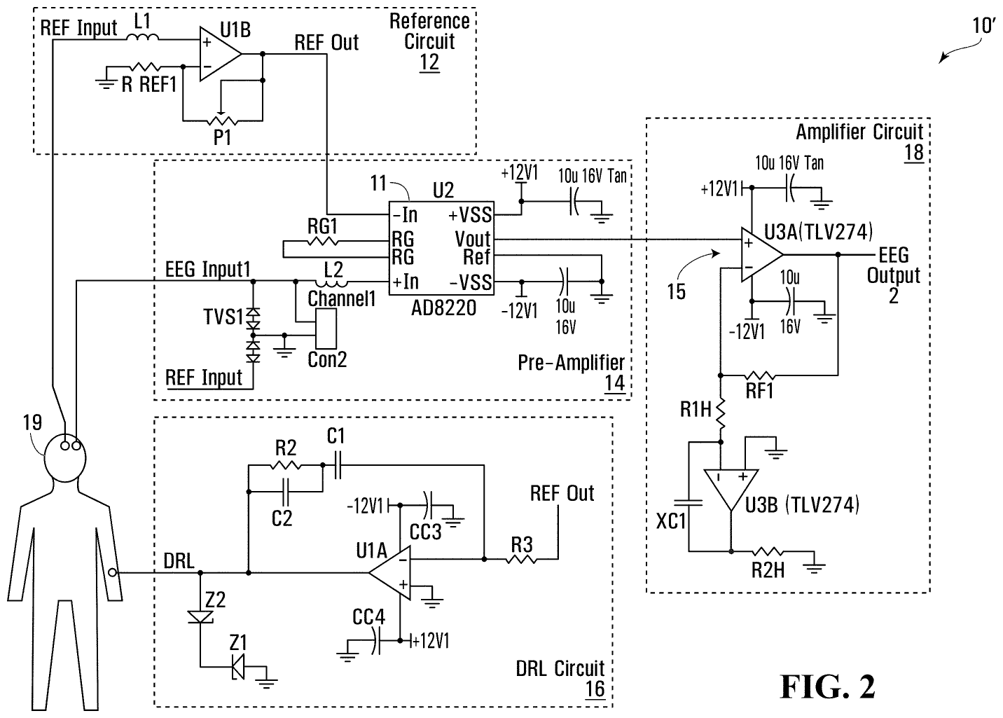

Now turning to FIG. 2, there is shown another embodiment of an EEG amplifier 10′. Amplifier 10′ is similar to amplifier 10, and therefore like elements are numbered using like reference numbers. Relative to amplifier 10, in amplifier 10′ high-pass filter 17 has been removed. Op-amp 15 is a non-inverting amplifier with an integrator on the inverting input. This integrator circuit avoids amplifier saturation caused by the electrode interface's DC offset since XC1 controls the amplifier gain according to:

ADIFFF(U3)=1+RF/(XXC1+(R1H+R2H)), where XXC1=1/j2πfXC1.

Therefore, the amplifier gain decreases as the frequency decreases. Although the low-frequency content has a lower amplitude, the ultra-low frequency waves are not eliminated.

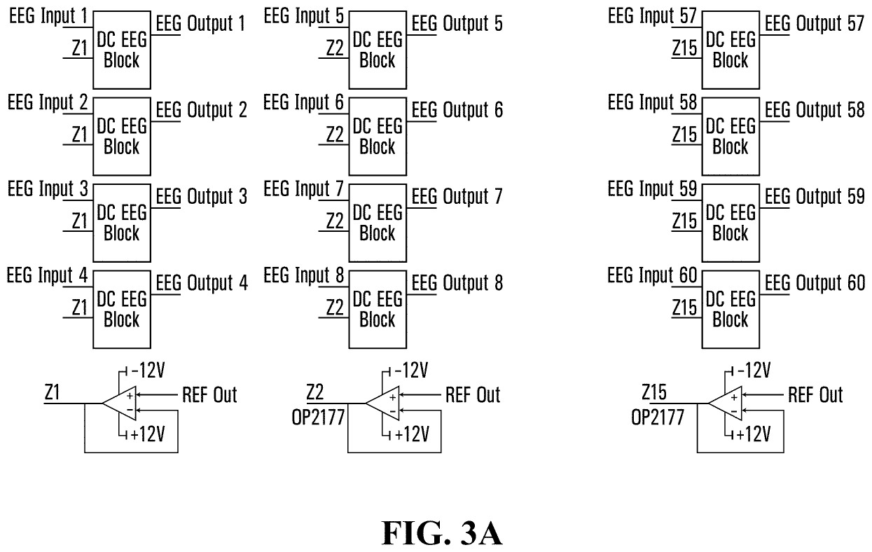

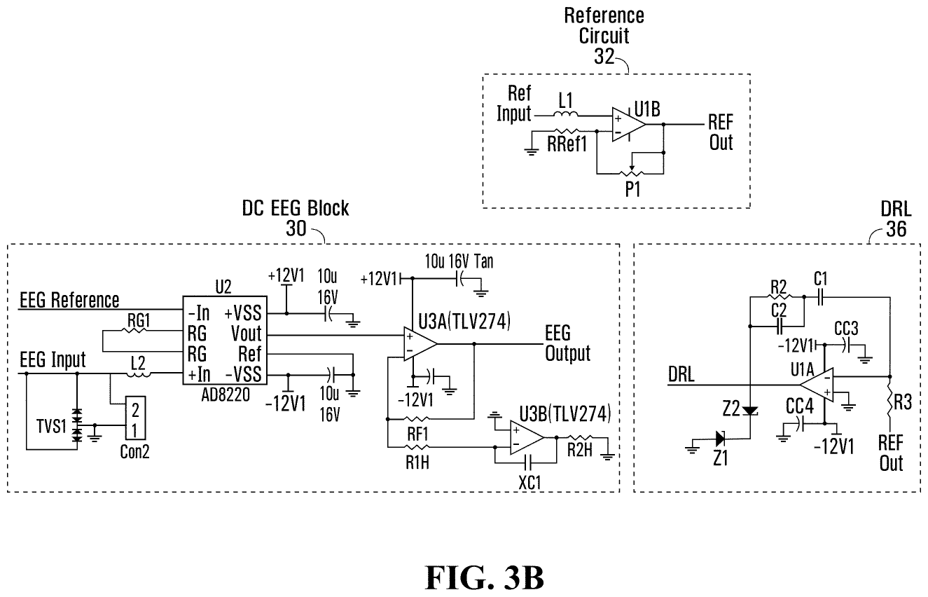

Turning to FIGS. 3A and 3B, there is shown a schematic circuit diagram of an EEG device having sixty channels, using sixty DC/AC EEG amplifiers (or “blocks”) 30, one DRL circuit 36, and one reference circuit 32. As explained above, one reference circuit is used (REF OUT) but the reference signal is buffered to avoid signal degradation loaded by sixty input channels. One buffer is used for every four DC EEG blocks 30, and so fifteen buffers are used for the sixty channels. For clarity, only channels 1-8 and 57-60 are shown in FIG. 3. As the skilled person would recognize, the disclosure extends to EEG devices comprising any suitable number of channels.

The components of FIGS. 1-3B have the following values. However, as the skilled person would recognize, the disclosure extends to embrace components with any other suitable values while still enabling DC/AC EEG device 10 to perform the functions and purposes set out herein.
R REF: 10 kΩ
P1:100 Ω
RG1: 2.61 kΩ
RG2: 100 kΩ
RG3:10 kΩ
L1, L2: FERRITE BEAD 220 OHM 0805 1LN
R3: 10 kΩ
R2: 500 kΩ
C1, C2: 1 nF
RH1: 1MΩ
CH1: 10 μF
TVS1: PESD5V0S2BT, 215
Z1, Z2: ZENER 8.2V
CC1, CC2, CC3, CC4, CC5: 10 μF, TAN
CC46, CC47: 100 nF
U1A, U1B, AMPA: TLV274
U2: AD8220

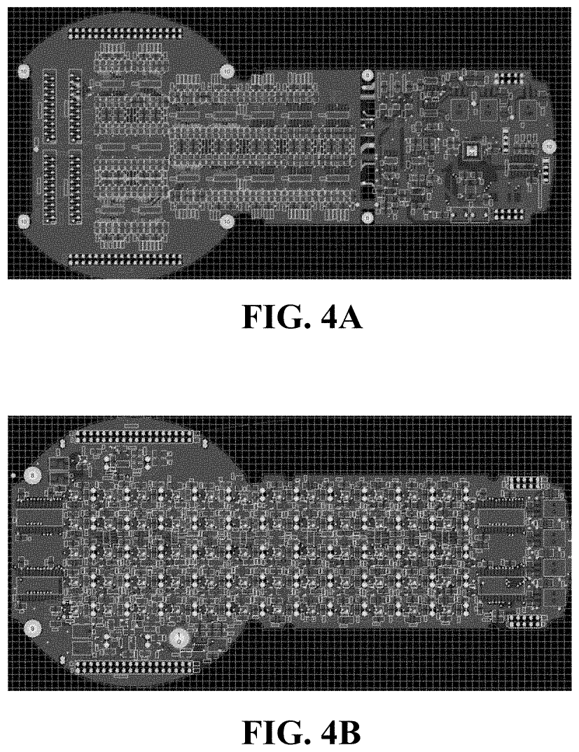

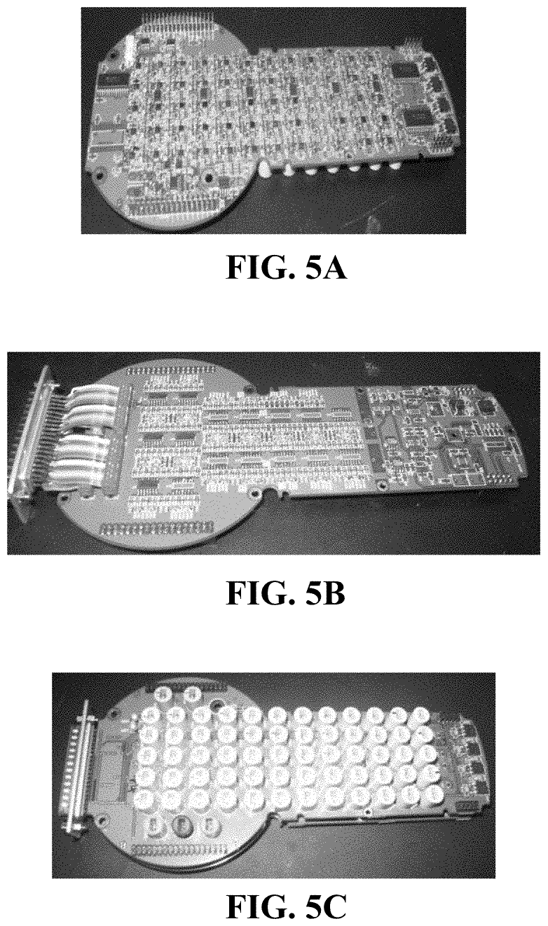

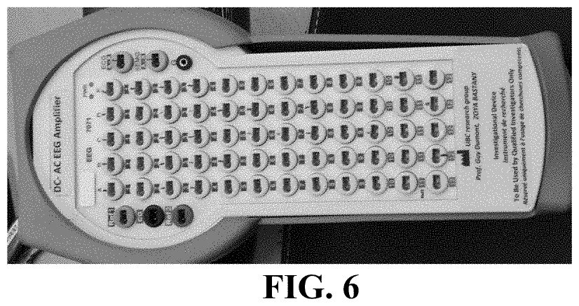

As can be seen in FIGS. 4A-5C, the EEG device comprises two PCBs each comprising six layers. The first board includes preamplifiers 14, reference circuit 12, DRL circuit 16, and input connectors. The input connectors are located on the bottommost layer, and the other components are located on the topmost layer. This aids to isolate the input signal lines from the other components. Preamplifiers 14 are positioned close to their respective input connectors to minimize common noise. DRL circuit 16, reference channel tracks, ground plate, IC power supply tracks, and output lines are located in the third, fourth, fifth, and sixth layers, respectively. The second PCB comprises amplifier circuits 18 and impedance checking circuits. Two-pin connectors were used for each EEG channel to connect the second pin to the ground plate. Thus, shielded wire could be used for electrode connections. The shielded wires greatly reduces ambient high-frequency noise. An embodiment of the DC/AC EEG device is shown in FIG. 6.

Thus, in one implementation of the above-described DC/AC EEG amplifier 10, any high-pass filter is eliminated from preamplifier 14. This enables amplifier 10 to detect frequencies below 0.02 Hz. The differential gain of preamplifier 14 is reduced to less than 200 to prevent amplifier saturation caused by the wide range of amplification (5 μV-30 mV) and high-pass filter elimination. The relatively high CMRR (>110 dB) at low differential gain may be obtained by decreasing the common voltage by minimizing the effect of resistor tolerance and driving the subject with a portion of the amplifier common voltage. The JFET input-instrument amplifier 11 increases the circuit input impedance to more than 10 GΩ, and its low bias current (<10 pA) assists with staying within the stable region of the electrode-gel-skin interface. Amplifier power consumption is small (1.3 mA/channel). Shielded cables, instead of typical EEG wires, are used. A very low-noise EEG analog-to-digital converter and power supply are also used.

Amplifier saturation in DC EEG may be caused by the electrode-gel-skin interface, the elimination of any high-pass filter in the preamplifier, and an amplitude range of from 5 μV (AC) to 30 mV (DC). Therefore, in some embodiments, the EEG device may address this by having a reduced differential gain (of about 20), a high-pass filter (with a cut-off frequency of 0.015 Hz) in the amplifier circuit, or an integrator in the amplifier circuit, and rail-to-rail op-amps in the preamplifier and amplifier circuit.

Low CMRR in DC EEG may be caused by higher electrode resistance, and a limitation on how much differential gain can be reduced, because of amplifier saturation. Therefore, in some embodiments, the EEG device may address this by using AgCl electrodes, using an amplifier with a CMRR of at least 110 dB, using a single resistance to set the gain, using a DRL circuit to decrease common mode voltage, using the PCB layout described herein, matching input impedance and capacitance, and decreasing noise by using shielded wires and having a two-pin input connector junction. Furthermore, a JFET input op-amp may be used to increase the amplifier input impedance to greater than 10 GΩ. The very low input bias current (no more than 10 μA) decreases the current density at the electrode-gel-skin interface.

### Design Verification in a Rat Study

Direct injection of potassium chloride (KCl) into the neocortex of rats stimulates the neuronal tissue to generate SD waves. Therefore, the simultaneous recording of DC and AC brain electrical activities was performed on the surface of the scalp and somatosensory cortex while 3M KCl was injected into a rat's neocortex. As further described below, the recorded negative DC potentials were evaluated based on their amplitudes, durations, and propagation velocities. Moreover, a variety of signal processing techniques aided in evaluating the initiated SD waves and discovering the role of signal morphology changes and propagation as a result of travelling from the neocortex to the scalp.

Twenty adult Wistar rats (210-400 g) were housed individually under controlled environmental conditions (12-hour light/dark cycles) with food and water available at libitum. The animals were anesthetized with intraperitoneal injections of chloral hydrate (350 mg/kg Sigma-Aldrich), and the head of each rat was placed in a stereotaxic instrument (Stoelting Instruments, USA). AgCl disc-recording electrodes (2.5 mm diameter with Ten-20 gel) were positioned on the skin above the somatosensory neocortex and connected through shielded wire to the AC/DC EEG device described herein.

Applying 3M KCl in the brain neocortex stimulates the brain neuronal tissue to generate an SD wave. A stylet was placed into the guide cannula to allow it to maintain patency. After withdrawing the stylet from the guide cannula, a 27-gauge injection needle was inserted. A polyethylene tube (Harvard Apparatus, Inc.) attached this injection needle to a 10 μL Hamilton syringe, and 10 μL of KCl solution was injected. To facilitate diffusion of KCl, the guide cannula retained the injection needle for an additional 60 seconds after injection.

The EEG device was made compatible with an EEG interfacing box and software (NRSIGN INC, Canada). The electrodes were connected to the EEG device via shielded wires. The interfacing included an analog-to-digital convertor (with sampling rates of 500-2,000 samples/s, and a resolution of 24 bits), a power supply, and a 50/60 Hz optional notch filter. The laboratory's temperature was set to a constant value of approximately 18° C.±5% to assist in achieving a low current density.

To estimate the normal EEG fluctuation from the baseline, mean and standard deviation of the signal were calculated for five seconds before the KCl injection, using Equation 3 and Equation 4, respectively.

$$\begin{matrix}μ = \frac{1}{N} {\sum }_{i = 1}^{N} {X}_{i} & Equation 3 \\ σ = {( \frac{1}{N} {\sum }_{i = 1}^{N} {( {X}_{i} - μ )}^{2} )}^{\frac{1}{2}} & Equation 4\end{matrix}$$

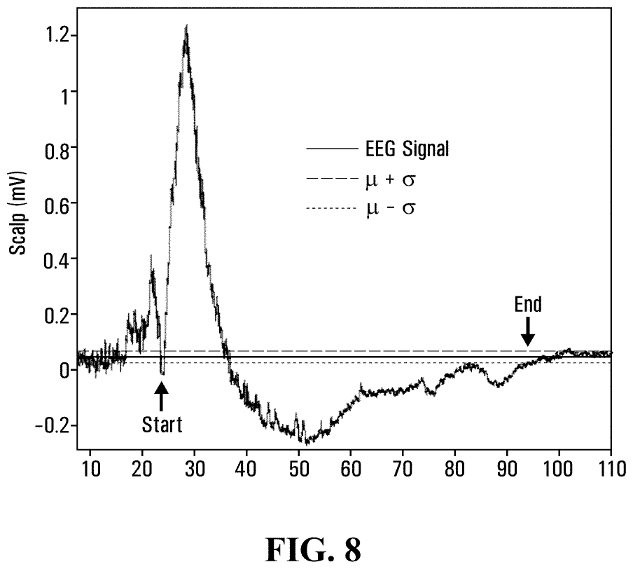

Xᵢ is the i^{th} sample of the signal, N is the number of samples within the 5 seconds, and μ and a are the mean and standard deviation of the signal, respectively. The recorded data was exported to MATLAB and the mean and standard deviations were calculated (FIG. 8). The start of the SD signal was defined as the moment that the EEG signal crossed one of the dashed lines and began to form the standard SD morphology (first or second component). The end of the SD signal was marked when the negative, low-amplitude component of the SD signal returned to the dashed lines. The SD signal amplitude was defined as the peak-to-peak voltage of the signal.

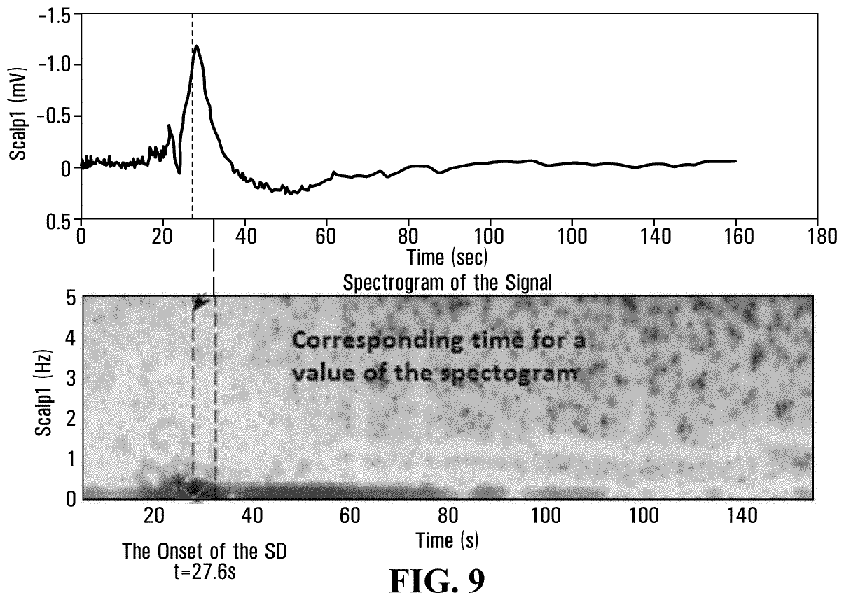

To determine the propagation speed, the following was performed. Each electrode has three coordinates defined as Horizontal (X), Vertical (Y), and Depth (Z). Since the Z-axis is zero for scalp electrodes, the distance between the electrodes was measured. SD has maximum energy in frequencies less than 0.1 Hz. Therefore, the maximum power amplitude of the SD was measured for frequencies of about 0 Hz, 0.05 Hz, and 0.1 Hz, using power spectrogram analysis (Equation 5). Then, the average of the corresponding times of these maximum values was measured (Equation 6). FIG. 9 illustrates the SD onset.

$$\begin{matrix}{t}_{f} = arg {max}_{t} {S}_{w} ( f , t ) & Equation 5 \\ {t}_{onset} = ( {t}_{f} ) & Equation 6\end{matrix}$$

(fᵢ) denotes the spectrogram of the SD wave, [symbol] [symbol] denotes the average symbol, and t_{f} denotes the spectrogram corresponding time. The time differences of SD onset were assumed to be the time delay between the propagated SD waves. According to the measured distance and the time delay, the propagation velocity was measured. The velocity from latency and distance between two electrodes was calculated. This calculation is based on the assumption that the SD spreads in parallel with the orientation of the electrodes.

To calculate the spectrogram of the signals, a defined window was moved through the signal. Equation 7 represents the windowed signal. The Fourier transform of the windowed signal was calculated using Equation 8, and the spectrogram of the signal can be determined by applying Equation 9.

x_{w}(τ)=x(t)*W(t−τ) Equation 7

X_{w}(f,τ)=∫_{-∞}^{+∞}x_{w}(τ)e^{-j2πft}dt Equation 8

(f,τ)=E_{f}[|X_{w}(f,τ)|²] Equation 9

The export of the EEG raw data occurred, without any filtering or modification from the EEG software to MATLAB, at a sampling frequency of 500 Hz. Applications of the 50/60 Hz notch and a low-pass filter (with a cut-off frequency range of 35-60 Hz) eliminated the unwanted noise. Equation 10 identifies the Kaiser window used in the spectral analysis.

$$w ( n ) = \{ \begin{matrix}\frac{{I}_{0} ( π a \sqrt{1 - {( \frac{2 N}{N - 1} - 1 )}^{2}} )}{{I}_{0} ( π α )} & 0 \leq n \leq N - 1 \\ 0 & Else\end{matrix} \qquad Equation 10$$

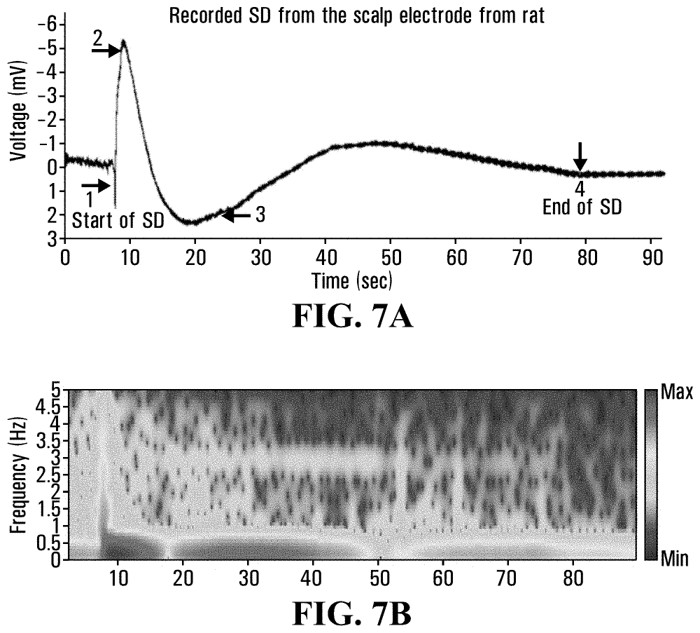

O₀ is the 0^{th} order modified Bessel function of the first kind, α is an arbitrary, non-negative real number that determines the shape of the Kaiser window, and N is the width of the window. All spectrogram analysis was performed with α=3 and N=100. Colours represent the energy of the signals within the frequency range of 0-4 Hz. The horizontal axis represents the time duration of the analysis, and the vertical axis represents the frequency range of the spectral analysis (see FIG. 7B).

Power spectral analysis loses phase information due to its second-order statistics from autocorrelation. Therefore, the EEG data estimated from the power spectrum includes minimal phase information of the original signal. Bi-spectral analysis is an informative, higher order statistical analysis and, unlike the power spectrum, it preserves Fourier phase information. Therefore, this method is capable of extracting the propagation effects of SD waves within the tissues. Moreover, using bi-spectral analysis allows for examination of the SD phases recorded from multiple electrodes and initiated from a common source. Equation 11 demonstrates the calculation of the bi-spectrum using the Fourier transform of a signal evaluated at f1 and f2.

$$B ( {f}_{1} , {f}_{2} ) = \frac{1}{k} {\sum }_{i = 1}^{k} Xi ( {f}_{1} ) Xi ( {f}_{2} ) {Xi}^{*} ( {f}_{1} + {f}_{2} ) \qquad Equation 11$$

X stands for the Fourier transform of x, and the non-stationary signal has been split into k time segments called epochs. Using convolution theory for the Fourier transform of x, the signal bi-spectral was calculated using Equation 12.

$${B}_{i} ( {f}_{1} , {f}_{2} ) = {\sum }_{{τ}_{1} = - ∞}^{{τ}_{1} = + ∞} {\sum }_{{τ}_{2} = - ∞}^{{τ}_{2} = + ∞} {C}_{i}^{3} ( {τ}_{1} , {τ}_{2} ) {e}^{- 2 π i ( {τ}_{1} {f}_{1} + {τ}_{2} {f}_{2} )} \qquad Equation 12$$

Cᵢ³(τ₁,τ₂) is the third-order cumulant of the signal. The cumulant can be computed by applying a natural logarithm to the moment, generating a function of random variables. For a stationary signal, the cumulant can be calculated using Equation 13.

Cᵢ³(τ₁,τ₂)=E{xᵢ(t)xᵢ(t+τ₁)xᵢ(t+τ₂)} Equation 13

E{ } is the statistical expectation. The equation clearly indicates that the cumulant depends on time lags, as the equation involves two shifted signals; the bi-spectrum provides a decomposition of the third moment. Therefore, the cumulant and corresponding bi-spectrum will be a 2D matrix. The cumulant matrix has symmetrical properties that are defined in Equation 14.

$$\begin{matrix}{C}^{3} ( m , n ) = {C}^{3} ( n , m ) \\ = {C}^{3} ( - n , m - n ) \\ = {C}^{3} ( - m , n - m ) \\ = {C}^{3} ( m - n , - n ) \\ = {C}^{3} ( n - m , - m )\end{matrix} \qquad Equation 14$$

For the bi-spectrum parameters, applying a window function to the non-stationary signal allows the defined samples to be selected. The window function must meet the symmetry properties of the cumulant provided in Equation 14 and must have the characteristics indicated in Equation 15.

w(m,n)=0 ∀(m,n)ϵC₃(m,n)=0

w(0,0)=1

W(ω₁,ω₂)≥0 ∀(ω₁,ω₂) Equation 15

w is the window function, W is the Fourier transform of w, and n and m are the number of samples along the vertical and horizontal axes. To simplify the calculation, the one-dimensional window of Equation 16 may be used.

w(m,n)=d(n)d(m)d(n−m) Equation 16

d stands for a one-dimensional window function.

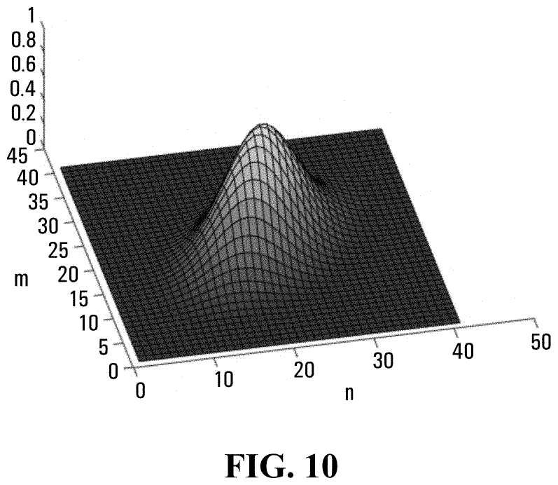

Within this study, the Parzen window (Equation 17 and FIG. 10) with 40 points was used.

$$d ( m ) = \{ \begin{matrix}1 - 6 {( \frac{\vert m \vert}{L} )}^{2} + 6 {( \frac{\vert m \vert}{L} )}^{3} , & \vert m \vert \leq \frac{L}{2} \\ 2 {( 1 - \frac{\vert m \vert}{L} )}^{3} , & \frac{1}{2} \leq \vert m \vert \leq L \\ 0 , & \vert m \vert > L\end{matrix} \qquad Equation 17$$

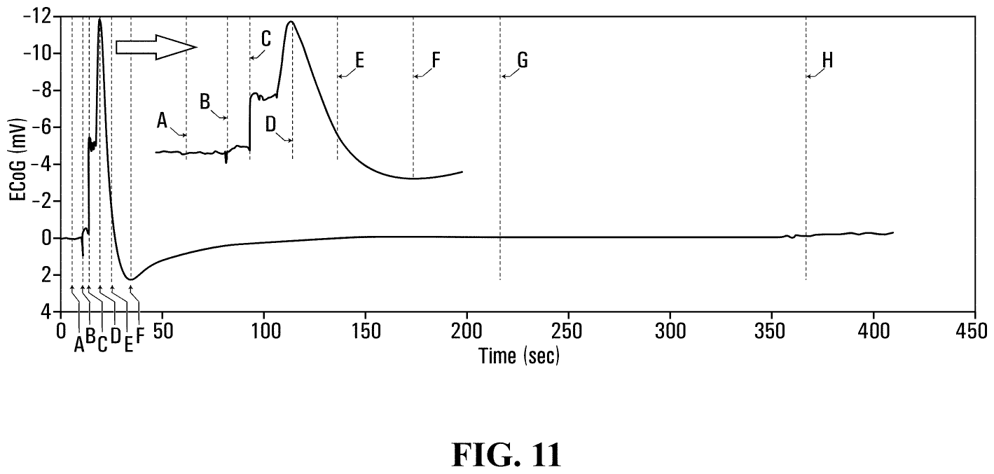

Particular critical instances in the SD wave were specified, including phases 1, 2, 3, depression and recovery states (as shown in FIG. 11).

FIG. 7A illustrates an SD wave (7.8 mV peak-to-peak and an approximate 72 s duration) captured from a rat's scalp when 3M KCl was induced on the rat's neocortex. The recorded SD wave was imported to MATLAB and filtered by a 3^{rd}-order Butterworth low-pass filter of 70 Hz and a 50 Hz notch filter without applying high-pass filtering. The recorded SD signal (FIG. 7A) displays the same features and shape as those of an SD signal recorded using an invasive method, such as ECoG. With respect to the reference electrode, the signal begins with a high-amplitude negative wave (phase 2) followed by a positive wave of smaller amplitude but longer duration (phases 3 and 4). At the beginning of the SD signal, a small positive wave (phase 1) is captured before the large, negative component. The signal has an amplitude of 7.8 mV and a duration of approximately 72 s. As can be seen, the amplifier described herein was able to amplify the ultra-low frequency signal well, and was not saturated by the electrode-gel-skin interface.

The signal's power spectrum (FIG. 7B) indicates that the signal has its highest energy at a frequency of approximately 0 Hz, when the SD wave is in phase 2 (at about the 10th second). The ultra-low frequency component still has high energy during most of the SD event and its value becomes small at the end of the SD wave (phase 4, at about the 80th second).

The amplifier described herein captured the field potential for a duration up to 80 s, from the surface of the scalp (FIGS. 7A and 7B). The amplifier was not saturated during the SD wave and all components of the wave passed through the electrode-gel-skin interface. Additionally, AgCl surface electrodes and a high concentration of Cl— in the electrolyte/gel are suitable for the recording of DC EEG.

Design Verification in Epileptic Patients

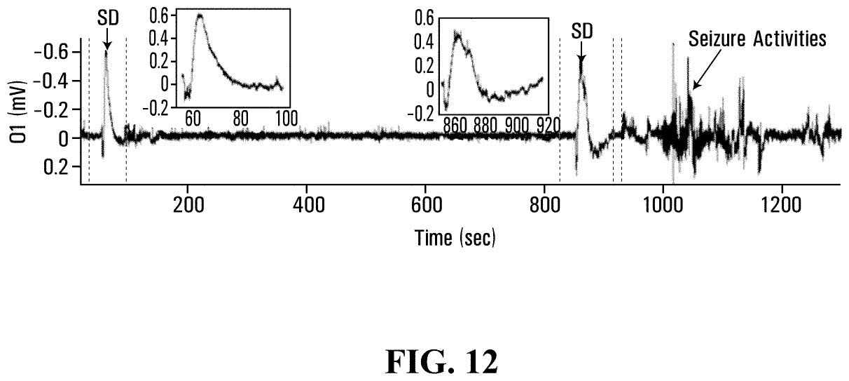

The AC/DC EEG device, the electrodes and the electrolyte were then verified by recording the EEG signal from the scalp of five patients with focal epileptic activity. FIG. 12 shows two SD waves captured on a patient's left occipital lobe (EEG of O1) within 12 minutes, with subsequent seizure activity. The first SD wave has an amplitude of 0.75 mV and a duration of 42 s. The second SD wave has an amplitude of 0.8 mV and a duration of 65 s, while the seizure activity frequency reaches 75 Hz with a maximum amplitude of 300 μV.

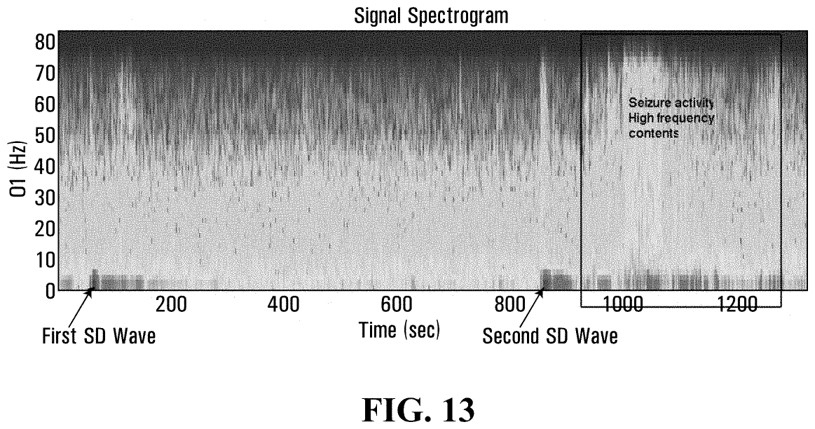

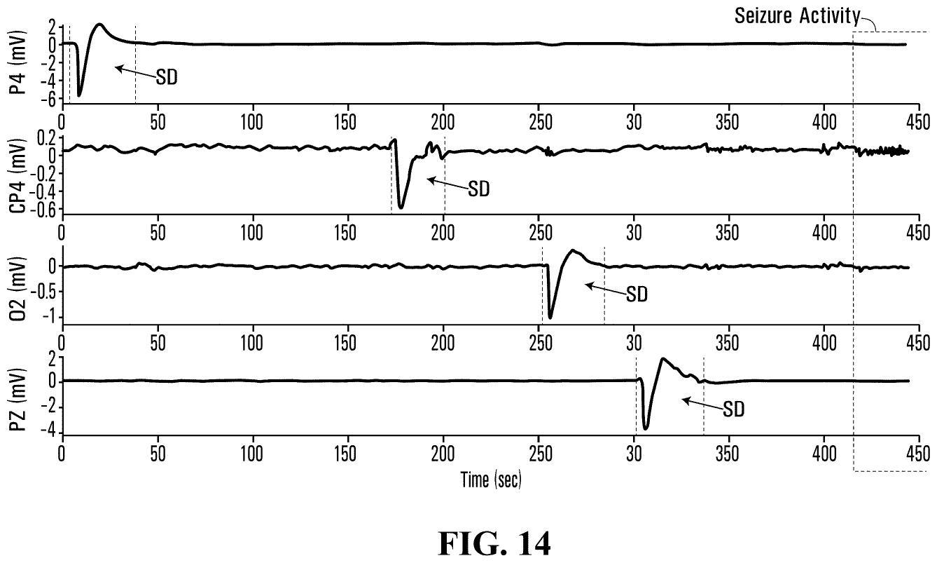

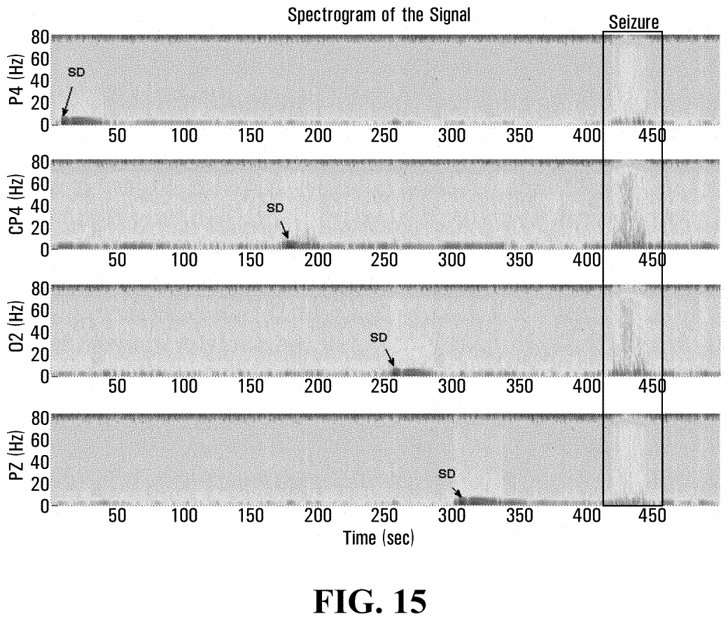

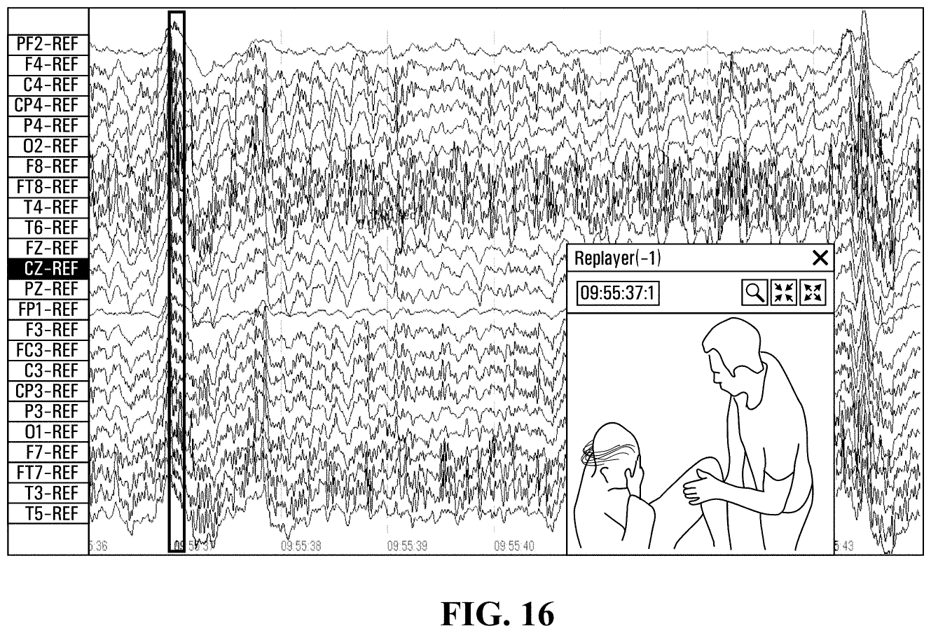

The signal power spectral analysis shows the high energy of the signal in the ultra-low frequency range (about 0 Hz) during the SD event, and high energy of the signal in the high-frequency range (up to about 70 Hz) during seizure activity (FIG. 13). The simultaneous recording of AC and DC EEG data, in sufficient quality, was therefore achieved. FIG. 14 shows the SD wave captured from the right parietal lobe of the patient's scalp, following which the SD propagates to the occipital lobe with an approximate velocity of 10 mm/minute. Moreover, a seizure with high-frequency content is captured afterwards (FIGS. 15 and 16). FIG. 14 also shows the AC and DC EEG recording. The spreading depression wave was recorded on the P4 electrode (Parietal lobe) with 8 mV amplitude and 38 s wavelength. This signal propagated to O2 and OZ with a propagation velocity of 11 mm/min. The epilepsy episode occurred about 370 s after the DC EEG pattern (SD wave) on P4. FIG. 14 shows that the amplifier can record DC EEG (SD wave) and high-frequency EEG (epilepsy episode) simultaneously.

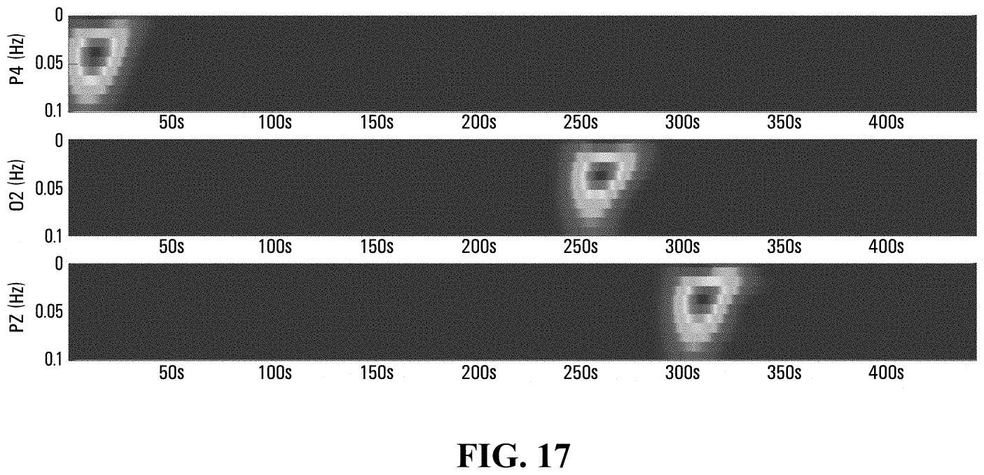

FIG. 17 shows the power spectrogram of the ultralow-frequency EEG (SD wave) of FIG. 14. In FIG. 17, the power spectrogram frequency resolution was increased in frequency to less than 0.1 Hz. This aids to have a better visualization of the ultra-low frequency contents of the SD wave.

Since DC EEG data has both high amplitude and duration, the sensitivity and time division by the EEG software needs to be rescaled to distinguish both types. In FIG. 14, the EGG device's sensitivity was adjusted to 300 mV/cm, and the time scale to 1 mm/s, to recognize the DC EEG. The EGG device's sensitivity was adjusted 50 pV, and the time scale to 30 mm/s, to recognize the AC EEG (FIG. 16).

The four propagated SD waves were analyzed using power spectral analysis. The first SD was captured on P4, having the maximum values of amplitude and duration between the other propagated SD with a voltage amplitude of 8 mV and a duration of 50 s. Moreover, the EEG device captured a seizure activity with higher frequency content (close to 70 Hz) approximately 2 minutes after the last SD event. The analysis indicates that the signals have the highest energy in frequencies less than 0.1 Hz during SD events, and have the highest energy in frequencies up to 80 Hz during the seizure event (FIG. 13). The results indicate ultra-low frequency (SD waves) and very high-frequency content were successfully captured.

### Combined NIRS and EEG Device

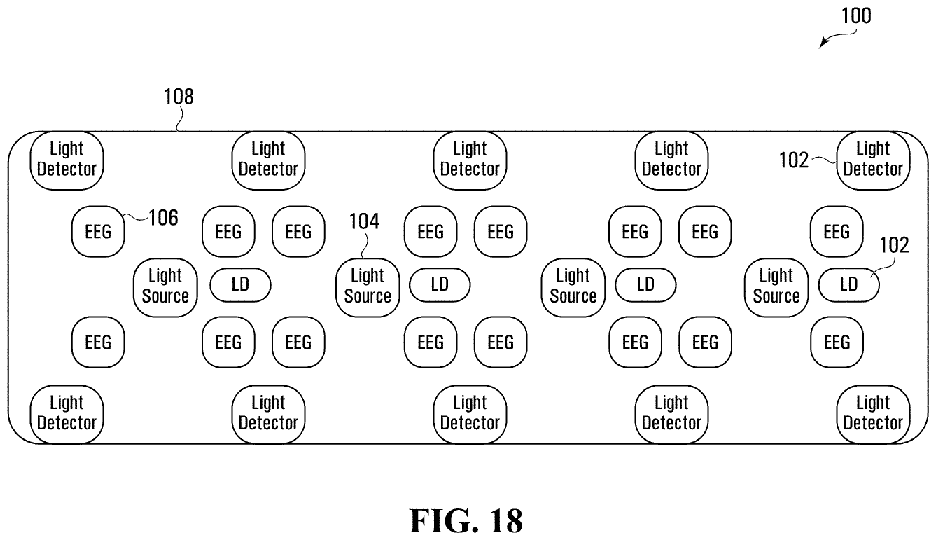

Now turning to FIG. 18, there is shown an embodiment of a combined near-infrared spectroscopy (NIRS) and EEG device (which may hereinafter be simply referred to as a “NIRS/EEG device”). NIRS/EEG device 100 comprises multiple light detectors 102 (which in the present embodiment are optodes), multiple light sources 104, and multiple EEG electrodes 106 (“electrodes” 106), located on a flexible, printed circuit 108. Electrodes 106 are positioned in-between optodes 102, to record the neurophysiological activity of the patient's neurons. Positioning electrodes 106 in-between optodes 102 maximizes the conjoined sampling area in the grey matter of the brain.

Optodes 102 are divided into “near” and “far” optodes, depending on their relative separation from the nearest light source 104. “Near” optodes monitor hemodynamic changes of solely the superficial layers of the brain, such as the scalp and skull, while the “far” optodes are only slightly associated with hemodynamic changes in the superficial layers of the brain and, more dominantly, with hemodynamic changes in deeper layers of the brain. Thus, by using mathematical processing (discussed below), hemodynamic changes in the superficial layers can be eliminated and concentration changes related to the deep layers of the brain can be estimated.

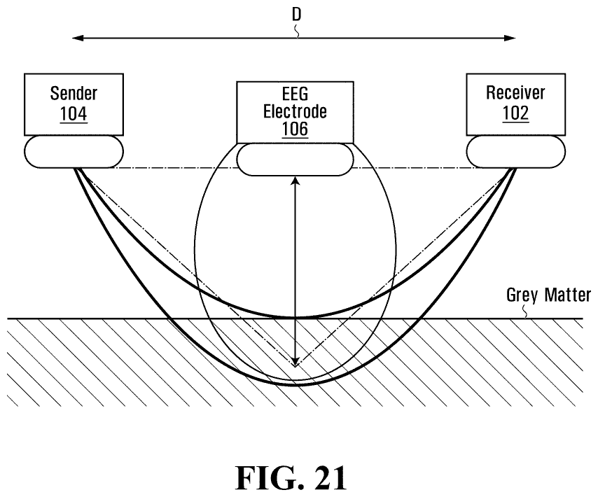

Light sources 104 emit near-infrared light that can penetrate the skin, scalp, and the external cranial and intracranial tissues of the human brain. As can be seen in FIG. 21, incident light crosses a banana-shaped pathway and approaches optode 102. The tissue chromophores absorb a portion of the emitted light. A portion of the emitted light may be scattered from its original direction. Usually, only the attenuation phenomenon is considered, and the scattering effect of tissues is neglected due to the high computation time and complexity needed for its analysis. The depth of penetration is roughly half the inter-optode distance.

Returning to the embodiment of FIG. 18, there are four light sources 104, with each source 104 configured to emit four different wavelengths of light (740 nm, 770 nm, 850 nm, and 870 nm), and with ten “outer” optodes 102 positioned around light sources 104. In this particular embodiment, the separation between outer optodes 102 is 28 mm, although the disclosure extends to any other suitable separation. The diameter of electrodes 106 (Cortech, AC-DC-AGE06, Ag/Cl) is 5 mm, although the disclosure extends to any other suitable electrodes. Flexible ribbon cables may be used to connect NIRS/EEG device 100 to the above-described amplifier and a light source controller. NIRS/EEG device 100 may then be secured on the patient's forehead by an elastic band.

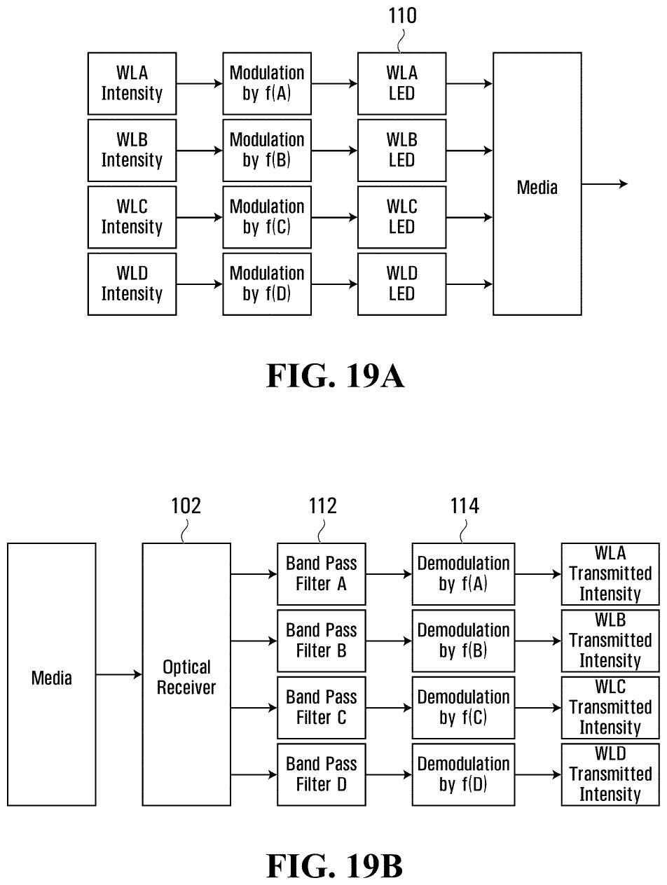

Using one or more light modulators (such modulators being known to those of skill in the art), the light from each light source 104 is modulated based on continuous wave amplitude modulation. In other embodiments, other suitable forms of modulation may be used, such as synchronous forms of modulation. Each emitted wavelength of light is modulated according to a particular carrier frequency. The carrier frequencies are sufficiently distinct from one another so as not to interfere with each other during transmission. FIGS. 19A and 19B show the continuous wave amplitude modulation developed for the NIRS/EEG design. WLA, WLB, WLC, and WLD represent the intensity of four different wavelengths for each light source 104. Specific carrier frequencies (shown as f(A), f(B), f(C), f(D)) modulate the intensity of each waveform. In one embodiment, the carrier frequencies are selected to be 1.5 kHz, 15 kHz, 54 kHz, and 85 kHz. A digital synthesizer (in one embodiment, AD9833: 24-bit D/A resolution, SPI interface, 0.004 Hz frequency resolution) is used to generate the cosine carrier signals.

$${I}_{0} {\sum }_{m = A}^{D} {I}_{m} {e}^{- i {ω}_{m} t} m \text{:} A , B , C , D \qquad Equation 18$$

In Equation 18, I₀ is the intensity of the incident light, Iₘ is the intensity of the corresponding waveform, ωₘ is the angular carrier frequency for the particular wavelength (A, B, C, or D), and t is time.

The modulated intensities are passed through LED current drivers 110 and subsequently emitted in the form of modulated near-infrared light toward the patient. A portion of the emitted light is reflected by the patient and detected at one or more of optodes 102. Based on the Beer-Lambert law, the transmission of light through a particular medium is modeled by a linear transformation, and the frequency of the applied optical signal does not change significantly during transportation. When considering the Beer-Lambert law and the linearity assumption for medium transmission, the transmitted signal, comprising the four modulated intensities, can be expressed by the following equation:

$${I}_{0} = {\sum }_{m = A}^{D} ( {I}_{m} {e}^{- i {ω}_{m} t} ) \cdot ( {e}^{{a}_{m} {c}_{m} ( t ) d} ) m \text{:} A , B , C , D \qquad Equation 19$$

I₀ is the intensity of incident light, Iₘ is the intensity of each wavelength, ωₘ is the angular carrier frequency for wavelength m, t is time, αₘ is the extinction coefficient of the compound, cₘ is the concentration of the absorbent compound, and d is distance.

I_{Total}=I₀+I_{AN(DC)}+I_{AN(ac)}+I_{AN(others)} Equation 20

In Equation 20, I_{Total} is the total light intensity that is delivered to the tissue. This intensity can be considered as being a summation of I₀, the modulated intensity of the NIR light sources 104, the low-frequency components of ambient light noise (such as sunlight and LED lamps), the AC component of ambient light in the power line spectrum, and other disturbing ambient light components. The summation of the four principal components detected at optodes 102 may be determined by the superposition theorem and the linearity of the light transmission function. The summation can be decomposed into its principal components by applying a bandpass filter to the received signal.

Bandpass filters 112 are used to decompose the received signal into four principal components and separate the signal from ambient interference. Each signal is then demodulated by demodulators 114 based on the particular carrier frequency that was used to modulate it prior to emission. The reflected intensity of the light may then be determined in order to estimate how much of the light was absorbed by the patient.

As shown in FIG. 19B, each optode 102 is associated with four bandpass filters. Therefore, for a device with ten receiver channels, forty bandpass filters are implemented. The design described herein uses a hardware implementation for the bandpass filters. One reason for opting for a hardware implementation is the number of channels that are used. The implementation of forty bandpass filters in software would be costly and time-consuming. Another motivation is the high sampling frequency. Modulation by the carrier frequency shifts the spectrum of the signal, and the sampling frequency needs to be at least twice the maximum frequency of the signal. Consequently, the sampling rate increases dramatically. To avoid a high sampling rate and associated computation time, an analog implementation of bandpass filters is preferred. However, if desired a software implementation may alternatively or additionally be used.

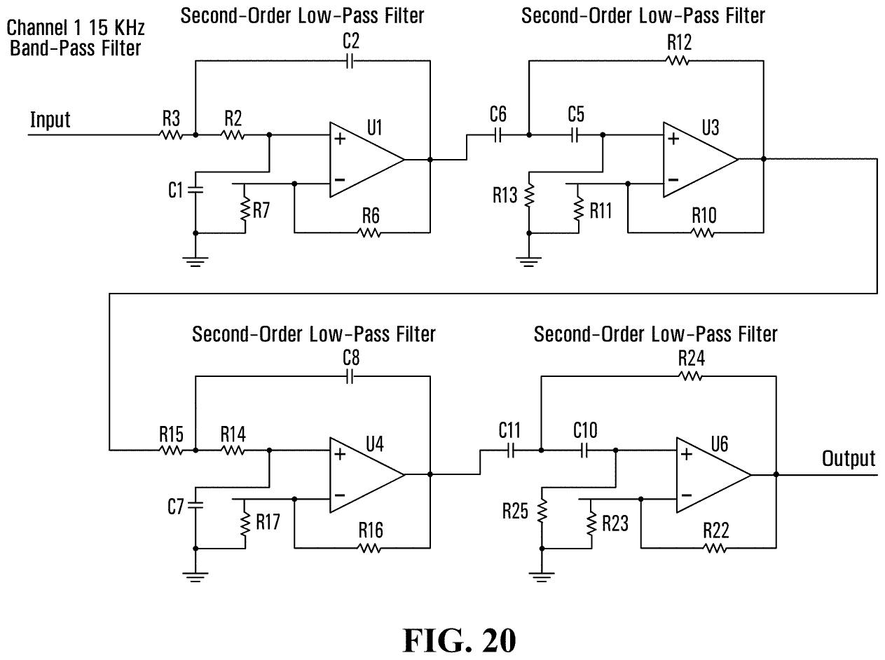

Second-order Sallen-Key low-pass and high-pass filters are used to implement a bandpass filter, as shown in FIG. 20. By using bandpass filters, the received signal is decomposed into its four principal components. Each component is related to a particular wavelength of light (WLA, WLB, WLC, or WLD). The bandpass filter also separates the signal from ambient interference signals. Ambient light interference may include interference generated from low-frequency light sources (e.g. sunlight, LED lightening lamps), 50 Hz lamps, and other electromagnetic interferences.

Amplitude Modulation

Theoretically, noise increases as the frequency of a signal decreases and approaches DC. In analog circuits, the noise level of an op-amp is proportional to 1/f, where f is input frequency. The light measurement may be disrupted by changing ambient conditions subject to noise. Therefore, choosing the measurement frequency to be away from the low-frequency noise improves the signal-to-noise ratio, allowing for the detection of weaker signals. Modulating the NIR light source signal at an appropriate frequency facilitates measurement of scattered light that would otherwise be buried in the noise.

An analog circuit implementation is used for implementing the NIRS frequency multiplexing technique described herein. As mentioned above, one advantage of analog implementation is its rapid processing time. Since the modulating carrier frequency in some channels may be 15 kHz, 67 kHz, or 300 kHz, digital sampling of the analog signal for numerical and software implementations needs to be remarkably faster in comparison to an analog implementation, in order for it to be effective.

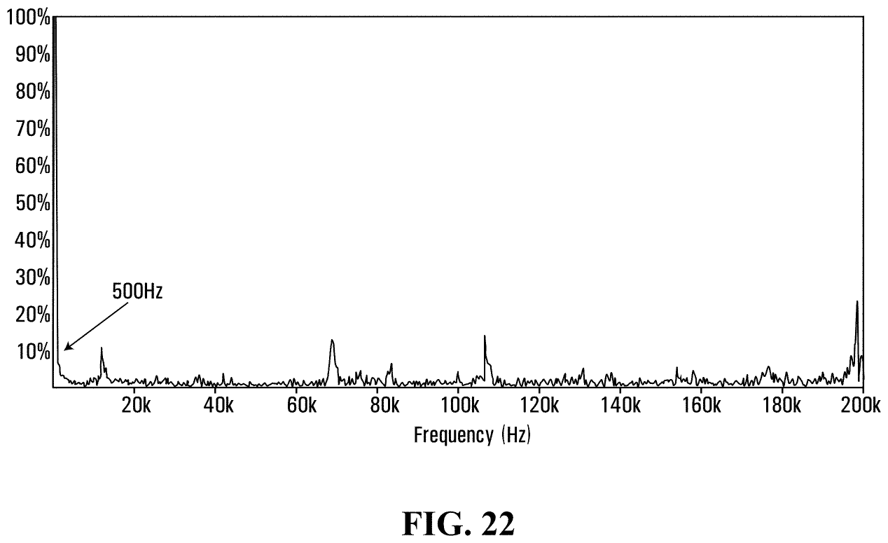

As also mentioned above, in the analog implementation of the NIRS frequency multiplexing technique, a Sallen-Key configuration was used to implement the bandpass filters. The Sallen-Key bandpass filter attenuates the effect of low-frequency ambient light noise (as shown in FIG. 22).

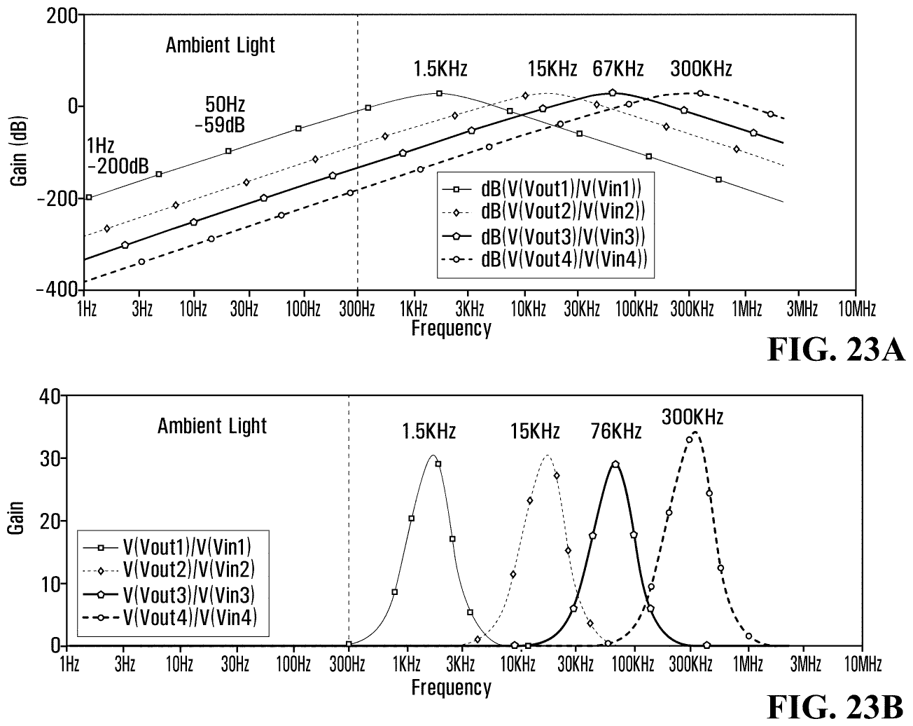

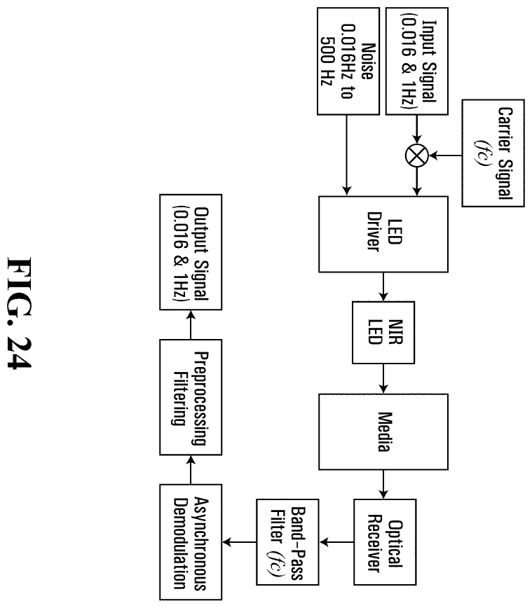

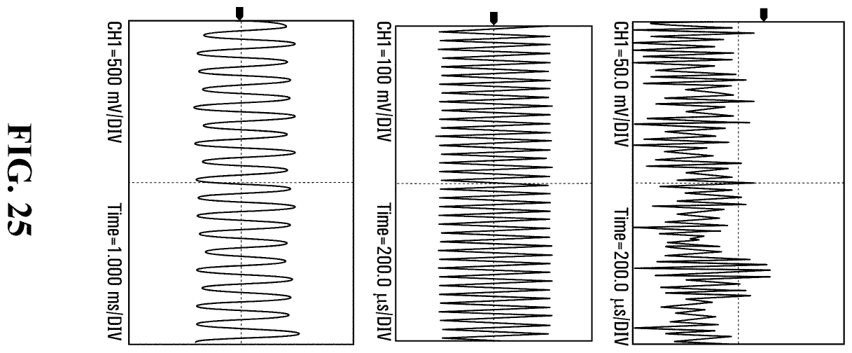

Numerical and experimental analysis was performed in order to validate the effectiveness of the NIRS frequency multiplexing technique. In the numerical method, the circuit was simulated by PSP ICE and ORCAD 2012. The frequency response of the circuit was estimated and is illustrated in FIGS. 23A and 23B. According to the numerical simulation, the minimum attenuation for 60 Hz is −59 dB and for 1 Hz is −200 dB.

For the experimental validation, the ambient light was simulated. Referring to FIG. 24, two known 1 Hz and 0.1 Hz sinusoidal signals were intentionally contaminated by simulated ambient light noise, and the effectiveness of the method of elimination of the ambient light noise was then evaluated. The ambient contamination factor was simulated using white noise with a frequency bandwidth between 0.016 Hz and 500 Hz. The intention was to compare the SNR of the output signal both with and without use of the modulation method.

The light source and the light detector were fixed on a synthetic medium made of multilayer transparent plastic. A microcontroller (ATXMEGA 128) was used to simulate NIR and ambient light. The signals (0.1 Hz and 1 Hz sinusoid signals) were synthesized, modulated and converted to voltage by using internal digital-to-analog converters. The signals were modulated using 1.5 kHz, 15 kHz, 67 kHz, and 300 kHz carrier signals. In order to simulate ambient light noise, white noise with a spectrum between 0.016 Hz and 500 Hz was added to the modulated intensity signal. The amplitude of the contaminated signal was adjusted properly to approach the desired SNR (50%) as an input signal.

Table 1 (below) shows the results of the measurement. The SNR of the signal was measured both with and without use of the modulation method. The SNR improves by increasing the modulation frequency. A dramatic improvement of SNR can be observed by using amplitude modulation. FIG. 25 illustrates the measurement that was conducted under real conditions. The position of the optodes was in the forearm. Two NIR sources modulated at different carrier frequencies were activated. The first signal shows the summation of two modulated NIR sources and ambient noise. The signal was input to bandpass filters and decomposed into two component signals. As shown in FIG. 25, SNR improved dramatically. Note that by using a combination of time multiplexing and amplitude modulation, the total optical energy can be controlled and reduced effectively.

| | | SNR of input signals | SNR Without | Amplitude modulation | | |
|---|---|---|---|---|---|---|
| Input signal | | simulated by | Amplitude | Mod | | |
| Frequency | | Microcontroller % | Modulation % | Frequency | | SNR % |
| 0.016 | Hz | ~50 | ~75 < SNR < ~75 | 1.5 | KHz | ~88 < SNR < ~92 |
| 0.016 | Hz | ~50 | ~75 < SNR < ~74 | 15 | KHz | ~89 < SNR < ~94 |
| 0.016 | Hz | ~50 | ~75 < SNR < ~82 | 67 | KHz | ~87 < SNR < ~93 |
| 0.016 | Hz | ~50 | ~75 < SNR < ~83 | 300 | KHz | ~88 < SNR < ~93 |
| 1 | Hz | ~50 | ~78 < SNR < ~84 | 1.5 | KHz | ~89 < SNR < ~92 |
| 1 | Hz | ~50 | ~78 < SNR < ~84 | 15 | KHz | ~89 < SNR < ~95 |
| 1 | Hz | ~50 | ~78 < SNR < ~84 | 67 | KHz | ~88 < SNR < ~93 |
| 1 | Hz | ~50 | ~78 < SNR < ~S4 | 300 | KHz | ~89 < SNR < ~93 |

Transmission of light at a particular wavelength within the tissue is determined by the combination of three major factors: reflectance, scattering, and absorption. Reflectance is a function of the angle of the light beam and the regularity and smoothness of the surface tissue. Reflectance decreases with increasing wavelength. Therefore, this effect is far less prevalent in NIR as compared to visible light. Scattering is mostly related to tissue composition and tissue interface layers. However, absorption is a function of molecular properties of substances within the light path.

The combined NIRS and EEG device 100 described herein uses a support 108 that may be placed on a patient's forehead. Electrodes 106 are placed in such a way that device 100 is able to record signals from the grey matter of the brain in the forehead. Increasing the inter-optode distance results in a greater sampling area, with incident light samples being obtained from deeper areas of the tissue. A greater sampling area comes at the cost of less spatial resolution. In addition, extending the inter-optode separation means that there is a longer path of sampling, and consequently less light energy reaches the photodetectors. Similarly, decreasing the inter-optode distance results in smaller sampling areas. Therefore, the incident light samples are obtained from more shallow areas, and spatial resolution improves. A benefit of a small sampling area is increased spatial resolution. Considering the interaction of inter-optode spacing, path length, spatial resolution, and the intensity of light at the receiver, there should be a trade-off among these parameters. A distance of 30 mm is proposed for inter-optode separation. The depth of penetration, empirically, is considered to be half the inter-optode distance.

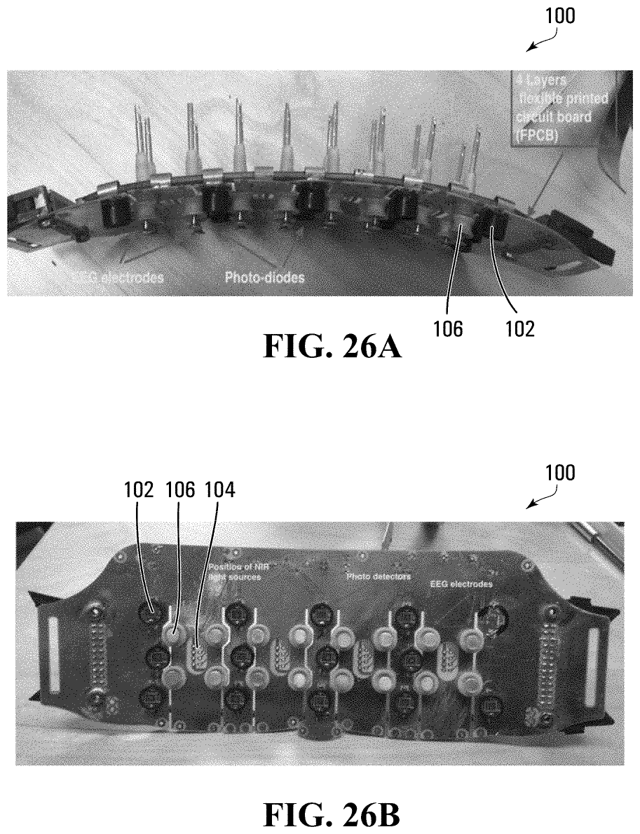

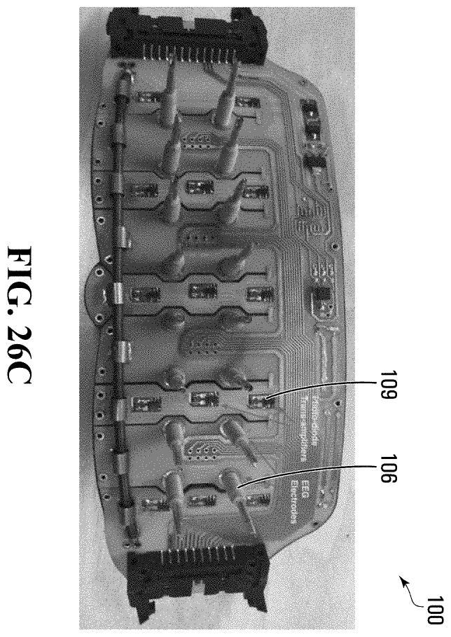

FIGS. 26A-26C illustrate NIRS/EEG device 100, showing optodes 102, light sources 104, electrodes 106, and optode trans-amplifiers 109.

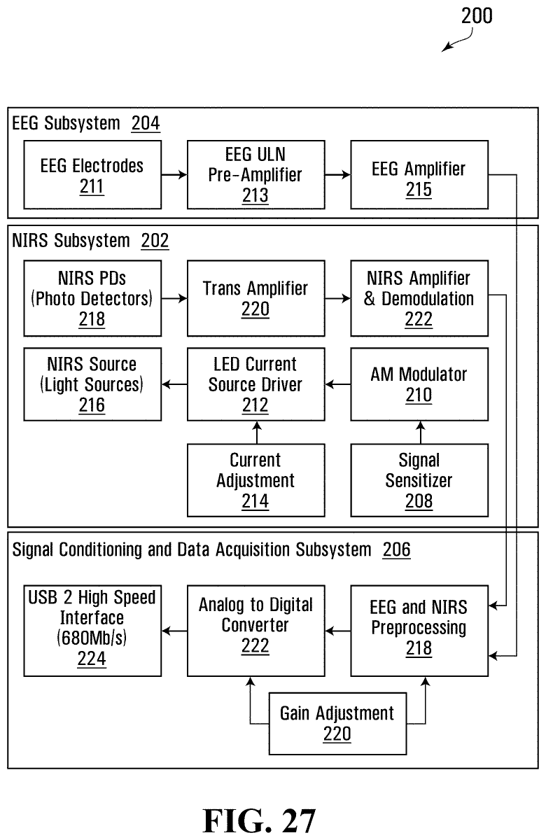

FIG. 27 is a schematic diagram of NIRS/EEG device 200 according to an embodiment of the disclosure. Device 200 includes NIRS subsystem 202, EEG subsystem 204, and signal conditioning and data acquisition subsystem 206. NIRS subsystem 202 includes signal sensitizer 208 configured to generate waveforms having specific wavelengths. AM modulator 210 modulates the amplitude of the generated waveforms according to one or more specific carrier frequencies. The modulated waveforms are passed through LED current source driver 212 which, with current adjustment 214, emits modulated NIRS light (light source 216). The modulated light, following reflection from the patient, is detected at NIRS photodetectors 218. The signal is then demodulated at NIRS amplifier and demodulator 222.

EEG subsystem 204 includes EEG electrodes 211, preamplifier 213, and EEG amplifier circuit 215. Preamplifier 213 and amplifier circuit 215 may be preamplifier 14 and amplifier circuit 18, as discussed above in connection with DC/AC EEG amplifier 10. After amplification of the DC component of the EEG signal by preamplifier 213 and amplifier circuit 215, the amplified EEG signal and the amplified NIRS signal are sent to EEG and NIRS preprocessing 218. The gain of the signals is adjusted at 220, and the signals are then passed through analog-to-digital converter 222 following which they are transmitted to a computer using for example a high-speed USB 2.0 (680 Mb/s) 224.

NIRS Recording During a Forearm Arterial Occlusion Test

Arterial occlusion was induced in the forearm of a patient by means of a pneumatic pressure cuff. The NIRS optodes were positioned on the forearm, below the pressure cuff to monitor the oxygenation of the arm. The occlusion was imposed on the forearm for approximately 45 seconds. A single NIRS probe was used during this experiment.

The probe was comprised of an LED emitting two wavelengths (λa=740 nm, [MTE1074N1-R, Marktech Optoelectronics] and λb=850 nm [TSHG6400, Vishay Semiconductors]) as the light source, and a photodiode (PDB-C160SM, 2.97 mm×2.65 mm, Luna Optoelectronics) as the light detector. The separation distance between the light source and light detector was 28 mm. The NIRS optodes were secured firmly to the brachioradialis, using an elastic VELCRO® fastening band.

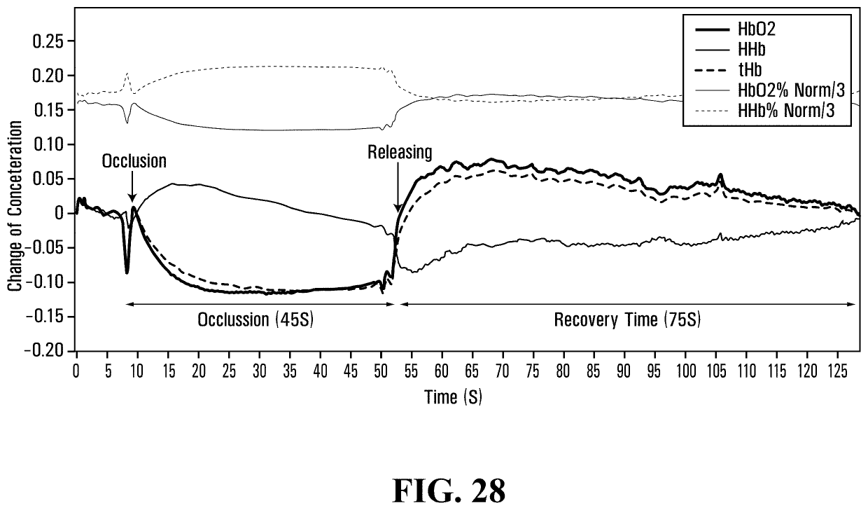

The objective of the experiment was to validate the functionality and effectiveness of the proposed method and its implementation, in measuring HHb and HbO₂ using NIRS. In this experiment, the hemodynamic response to an arterial occlusion of the forearm of a healthy male subject was investigated. This procedure is a straightforward trial for the validation of NIRS instruments. The gradient of the chromophore variations, in this instance, is associated with local tissue oxygenation. When the cuff is released, the changes are reversed. A hyperemic response is observed due to auto-regulative mechanisms. In comparison to measurements taken from the brain, the absorption of light was higher; this higher absorption rate seems to be due to the presence of different types of tissue chromophores. As shown in FIG. 28, the occlusion caused a very tiny artifact and was 45 seconds in duration. Upon release of the cuff, the HbO₂ and HHb returned to their original levels which is shown as the recovery time of 75 seconds in FIG. 28.

Hemodynamic and Electrophysiological Response to Repeated CPT

In this experiment, NIRS and EEG signals were recorded concurrently during a cold pressor test (CPT). The device was comprised of two subsystems: EEG and NIRS. The NRSIGN EEG interfacing box and its accompanying software (NRSIGN, BC, Canada) were used. Low profile Ag/AgCl (Cortech, AC-DC-AGE06, diameter ˜5 mm) electrodes were chosen for the recording of EEG signals. The NIRS hardware was implemented by using the amplitude modulation technique for the purposes of eliminating the effect of ambient light. The NIRS subsystem comprised of a light source controller, amplifier, and optodes. The light source controller was responsible for modulating the intensity of the light, and adjusting the radiated energy levels between 0.5 mW and 1.5 mW. The amplifier comprised a trans-amplifier, demodulator and analog filters. The designed NIRS/EEG forehead probe (as seen in FIGS. 26A-C) was used. The light sources were LEDs emitting 740 nm (MTE1074N1-R, Marktech Optoelectronics) and 850 nm (TSHG6400, Vishay Semiconductors) light. These LEDs were integrated into a single package. The light sensors were PDB-C160SM, by Luna Optoelectronics. The probe is relatively flexible and can easily be attached to the subject's forehead. The probe cap comprises of i) four NIRS light sources, each configured to emit 740 nm and 850 nm light; ii) ten NIRS “far” light sensors with an inter-optode separation of 28 mm; iii) four “near” light sensors with an inter-optode separation of 12 mm; and (iv) sixteen Ag/AgCl EEG (diameter ˜5 mm) electrodes positioned in-between the “far” NIRS optodes. Two ribbon cables connect the probe to the amplifier and light source controller.

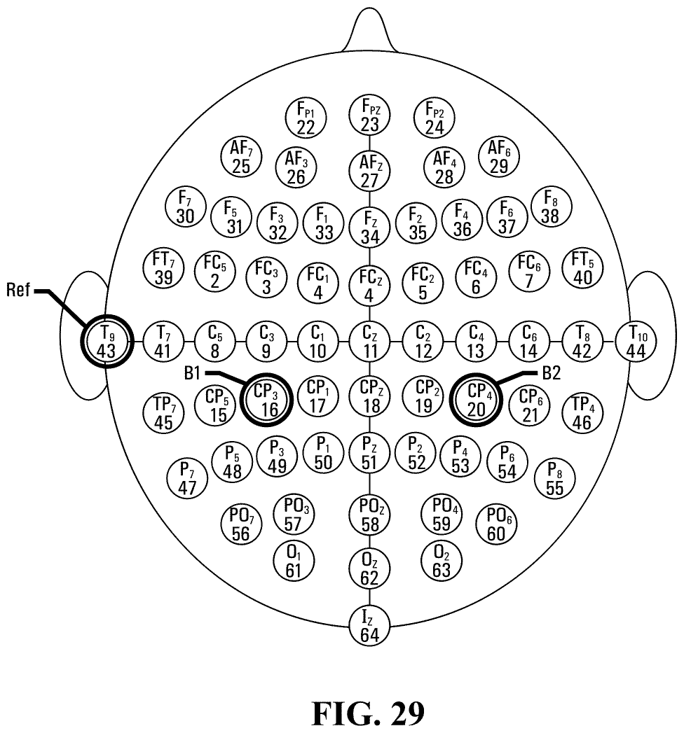

As shown in FIG. 29, two additional EEG electrodes (Cortech, AC-DC-AGE06, diameter ˜5 mm), B1 and B2, were positioned on the portion of the probe that overlies the sensory cortex. A reference electrode was positioned on the left ear. An Ag/AgCl electrode (Ambu® Neuroline 700, disposable solid gel electrode) was used as a reference electrode (on the left (43) ear and FZ (23) according to 10/10 EEG electrode placement).

This study was an observational study, and five healthy volunteers between 20 to 50 years of age, male and female, were enrolled. All participants gave written, informed consent to participate. The subjects were seated comfortably in armchairs. The probe was fastened symmetrically to the forehead of subjects as previously described. The optical density at two wavelengths (740 nm and 850 nm) as well as the EEG signals were sampled at a rate of 512 samples per second. Conductive paste (Ten20, Weaver) was applied to the EEG electrodes, in order to minimize the skin-electrode impedance. The experiment was conducted at room temperature (22-24° C.) and with normal ambient light presence. The relative changes of HHb and HbO₂ and total Hb were measured as hemodynamic variation parameters. The EEG signals were recorded concurrently. Two EEG-reference electrodes were used; one positioned frontally Z (23, 10/10 EEG electrode system), and the other one on the left ear (43, 10/10 EEG electrode system). The experiments began with two minutes of baseline recording, followed by immersion of the left hand up to the wrist in ice water (˜0° C.) for 120 seconds, followed by 120 seconds of recording at rest, after removal of the limb from the ice water (22-24° C.).

The cortical processing of the tonic pain caused by CPT has been studied in some neuroimaging studies. Several brain regions have been shown to be involved in the processing of noxious cold stimuli. The objective of the experiment was to assess the functionality of the NIRS-EEG device in recording the cerebral hemodynamic changes in response to CPT. The test was designed to confirm the results and check the functionally of the proposed method by using the implemented prototype and comparing its results with a reference device, under similar conditions. Ambient temperature and other external stressors are known to influence heart rate (HR) and blood pressure (BP). The cold pressor test (CPT) is a conventional procedure, broadly used across research disciplines involving psychological, cardiovascular, and neurological disorders. Abrupt and increasingly uncomfortable cold stress, as imposed during CPT, induces the extensive activation of the sympathetic nervous system and discharge of norepinephrine. These responses combine to increase BP. By increasing BP, we expect both THb and HbO₂ levels to increase in the tissue of the forehead.

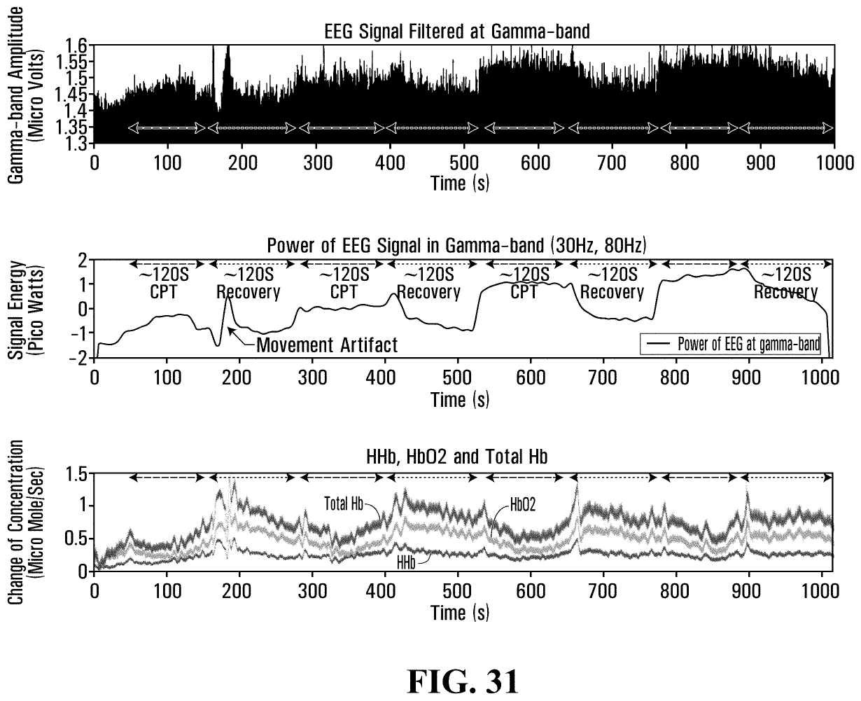

The HHb, HbO₂, and total Hb were estimated based on the modified Beer-Lambert law. Motion artifacts were found to be a barrier to the successful analysis of results in one case. However, in four subjects the results were very similar, reproducible and could be considered for further analysis. The optical density of the tissue in the forehead area is illustrated in FIG. 31. In this experiment, R and IR represent the optical density of tissue at 740 nm and 850 nm. Two wavelength intensities were modulated using the source controller. The instrument measured the hemodynamic response during both CPT and recovery time.

The hemodynamic variation of the brain is illustrated in FIG. 31. The HbO₂ concentration increases during the CPT test and decreases during the recovery period. In the recovery period, the subject's hand was exposed to room temperature (22-24° C.). The concentration of HHb also increases slightly during the cold pressor stimulus, while the HHb and HbO₂ both return to the baseline during the recovery period.

The response of the “near” and more superficially penetrating optodes (D=12 mm) was very similar to the “far” optodes (D=28 mm). The signals produced by the optodes from the left and right sides of the probe were compared, and no significant differences were observed between the both sides.

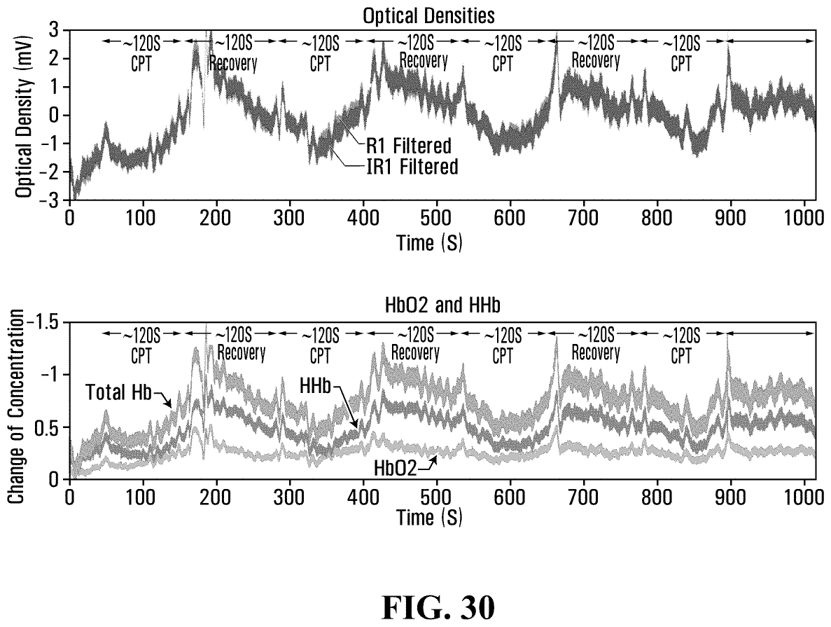

Based on these results, the functionality of the device appears acceptable and the instrument is able to detect variations in HHb and HbO₂ concentrations. After investigating four subjects, no meaningful differences were found between symmetrical NIRS optodes (left side and right side). Moreover, it seems that the increasing of total Hb in these subjects is related more to increasing the volume of the blood in the tissue according to the increased blood pressure during CPT (as shown in FIG. 30).

Hemodynamic Change and EEG Activity Response to CPT

Pain may be characterized as an unpleasant experience that affects the conscious awareness of noxious sensations. The measurement of physiological responses to pain may allow the quantification of pain levels. Pain assessment can be critical, especially in patients with chronic disorders of consciousness. These patients have a limited ability to communicate; in spite of their low-level consciousness, somehow they are able to process the pain signals that are generated by peripheral sensory nerves. Several researchers have investigated the neural signature of pain awareness and perception and its relation with Gamma-band oscillations (GBO). The perception of pain invokes a vitally protective reaction, urging the subject to react. Painful stimuli induce widespread propagation of cortical excitability and alerting signals that facilitate a swift reaction to the stimulus. Gamma-band oscillations may be related to voluntary selection, enhanced visual and auditory recognition processes, or tactile perceptions. Additionally, Gamma-band oscillations can be observed in response to pain stimuli. During a painful stimulus, the induced Gamma-band oscillations are proportional to the amount of pain consciously perceived. According to recent investigations, the sensory cortex in the central part of the brain as well as the prefrontal lobe may be most significantly implicated in gamma-band oscillations.

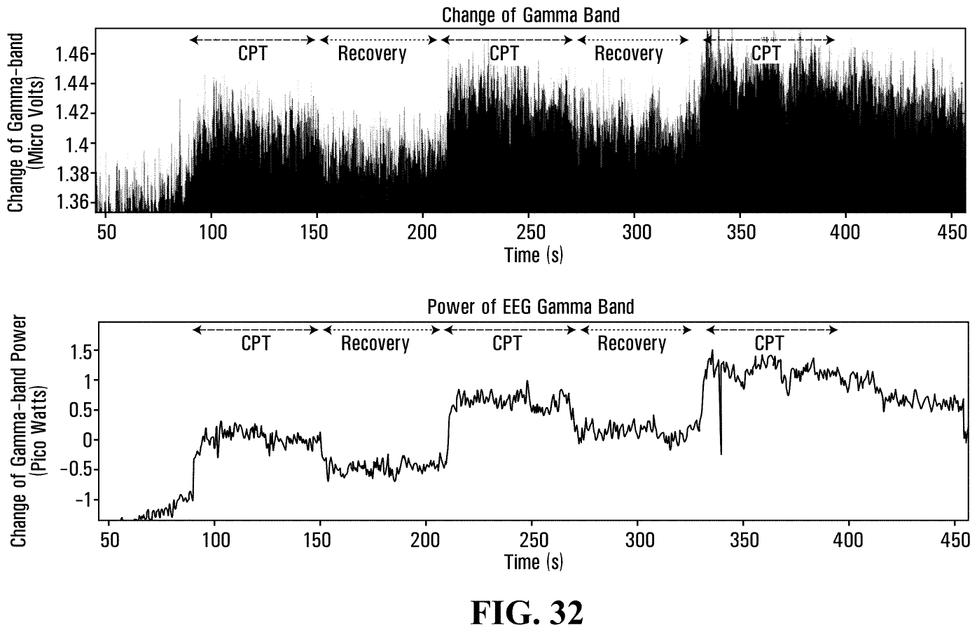

In this part of the experiment, it was intended to measure EEG and hemodynamic responses to the painful stimulus produced by the CPT. The position the EEG electrodes and NIRS optodes was similar to the experiment described above. The EEG signal recorded from the electrodes over the frontal lobe was filtered in the Gamma band (30 to 80 Hz). The Gamma-band oscillations, condensed in the time domain, are illustrated in FIG. 31, top panel. As shown, the Gamma-band oscillations increased during the CPT and decreased during the recovery time. The energy of the Gamma-band is computed in the second panel of FIG. 31. The energy signal is smoothed by using a Hanning window. The energy of the EEG signal in the Gamma band increased during application of the CPT test. The activity of neurons in the Gamma band can be due to the pain that is caused by the CPT (as shown in FIG. 32).

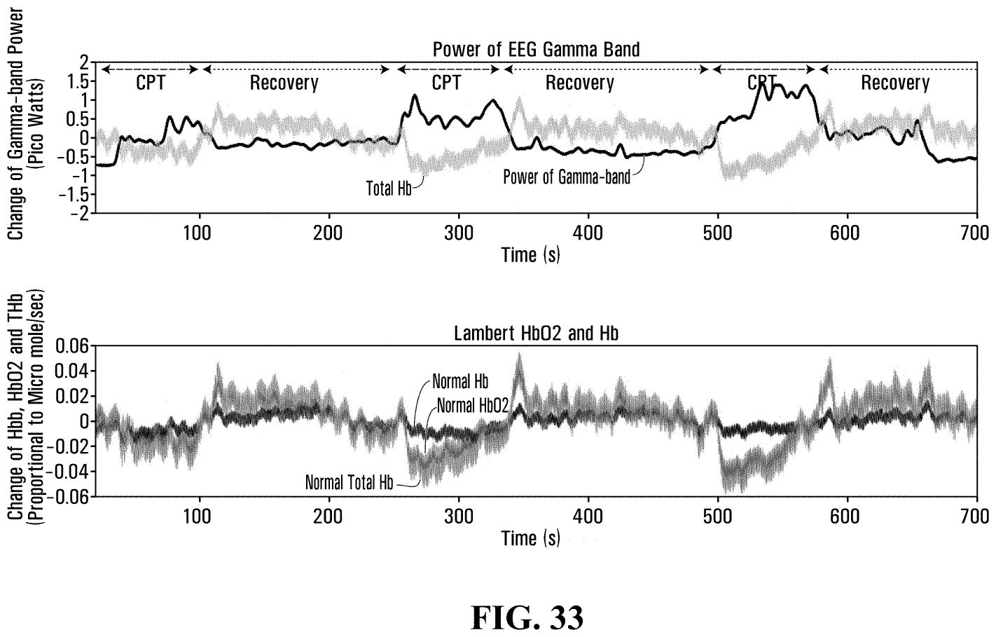

In this experiment, we investigated the presence of the Gamma-band during the CPT in the area of the brain which is near to the sensory cortex (B2). As is illustrated in FIG. 33, the power of the Gamma-band increases in the sensory cortex during CPT. Additionally, the power of the Gamma-band appears to be correlated with total Hb of the tissue in the forehead.

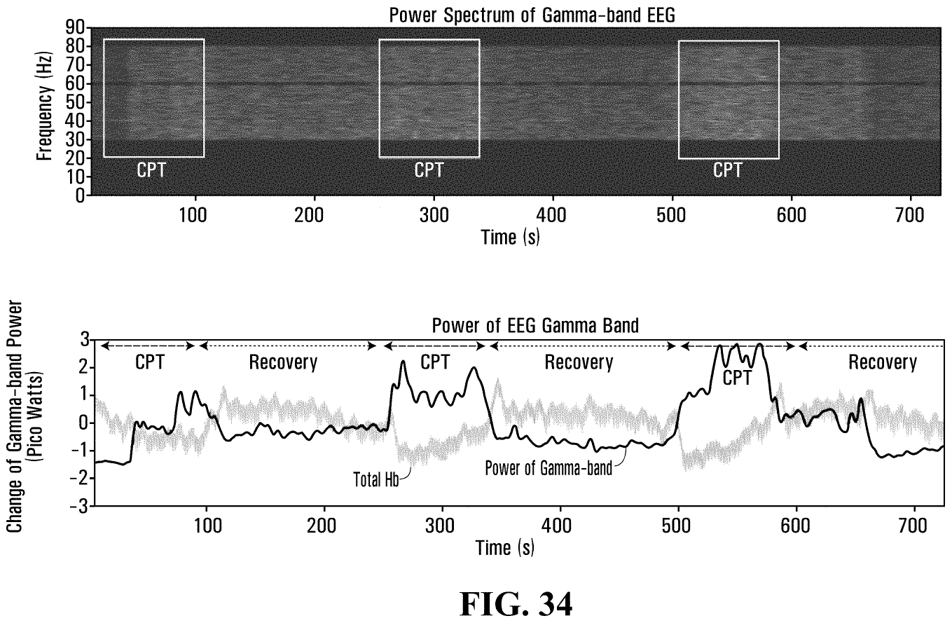

FIG. 34 illustrates the STFT (Short Time Fourier Transform) of the EEG signal in the Gamma band. The distribution of Gamma-band oscillations, induced by CPT, is computed over the 30 Hz to 80 Hz frequency bandwidth. The signal is recorded from B2, which is near the sensory cortex.

The correlation between total Hb and the power of Gamma-band oscillations was examined in four subjects. In each subject, the correlation between total Hb (forehead, right side) and the power of the Gamma-band oscillations (forehead, right side) in-between corresponding electrodes were calculated. A significant correlation (P-value <0.05, 95% confidence interval) was observed between these two measurements. In addition, the correlation between total Hb (right side, forehead) and the power of the Gamma-band oscillations in the sensory cortex (B2 electrode, right side) was examined in four subjects. A significant correlation (P-value <0.05, 95% confidence interval) was observed. Correlative analysis demonstrated a strong association between total Hb and the power of the Gamma-band oscillations in localized (forehead, left side) as well as general (sensory cortex) areas.

To summarize, the increase of blood perfusion during CPT was observed in all areas of the forehead. Additionally, detection of Gamma-band oscillations was conducted. Brain activation is accompanied by a complex sequence of cellular, metabolic, and vascular processes. Neuronal activity is an energy-consuming process that necessitates a high amount of glucose and oxygen and, consequently, produces a large amount of CO₂. A regional increase in cortical activity accompanies a local increase in blood flow. The results show an increase in Gamma-band activity and total blood perfusion (30-80 Hz) over frontal scalp sites, due to the subjective experience of pain. A high correlation between the power of the Gamma band (30 Hz to 80 Hz) and the total Hb was observed. The experiment was important to demonstrating the significance of NIRS/EEG technology in neurology and neuroscience applications.

Hypoxic Breathing Test

Conventional ideas in EEG consider most of the electrical signals gathered on the surface of the scalp to be produced by neuronal networks on cortical dipoles. Some investigations have confirmed the link between glial cells and the creation of slow local potentials throughout spreading depression, seizures, and sleep. Furthermore, studies in the early 1950s-1970s concluded that slow potential shifts are produced at the cerebrospinal fluid interface. However, the correlation between slow-EEG or DC-EEG and brain oxygenation has been the subject of few investigations. Some of this correlated activity may be caused by low-frequency neuronal activity. The neurovascular coupling mechanism reacts to the high concentration of CO₂ (pCO₂) and causes increased oxygenated blood perfusion to the tissue. However, the neurovascular coupling is only slightly sensitive to changes in oxygen concentration (pCO₂). According to recent investigations, changes in pCO₂ may cause low frequency shifts, indicated by very slow-EEG signals. However, these slow-EEG signals do not necessarily represent neuronal activity. The non-neuronal slow-EEG shifts caused by pCO₂ changes have been investigated in animal subjects.

Cortical spreading depolarization (CSD) is a self-propagating signal, caused by transient loss of neuronal transmembrane ion gradients. CSD is associated with vast and dramatic variation in cerebral blood flow (CBF). Despite the initial increases in CBF, hypoxia may occur and extend far into territories of capillary supply. This inverse hemodynamic response is due to swelling of neurons during severe CSD, and may delay the high-energy consumption activity of neuronal recovery. The swelling of tissue during recovery is the major cause of hypoxia; however its effect is superimposed on the highly energy-dependent activity of neuronal recovery.

In this experiment, the dependent relationship between the related physiological parameters of brain tissue, oxygenation, and slow-EEG potentials was examined by non-invasively recording NIRS and EEG during hypoxic breathing. Hemodynamic changes in the forehead tissue were measured by using NIRS alongside concurrent measurements of slow-EEG potentials produced by the brain tissue. During the experiment, an altitude simulation kit was used to restrict the concentration of oxygen in the air that the subject inhaled. During use of the altitude simulation kit, arterial oxygenation, as measured using a pulse oximeter on the index finger of the subject, dropped dramatically. This confirmed that hypoxic conditions had been successfully achieved.

The NIRS hardware that was used is the same as previously described. In addition to the 16 EEG electrodes, two additional EEG electrodes (Cortech, AC-DC-AGE06, diameter ˜5 mm), B1 and B2, were positioned on the portion of the probe that overlies the sensory cortex. A reference electrode was positioned on the left ear. An Ag/AgCl electrode (Ambu® Neuroline 700, disposable solid gel electrode) was used as a reference electrode (on the left (43) ear and FZ (23) according to 10/10 EEG electrode placement). A pulse oximeter on the right index finger (Masimo SET® Rad-8 Pulse Oximeter, Masimo Inc.) monitored arterial blood oxygen saturation. The altitude simulation kit used to create hypoxic breathing conditions was the AltoLab Platinum BOOST from AltoLab Inc.

Five healthy volunteers between 20 and 50 years of age were enrolled in the experiment. All subjects provided written, informed consent to participate in the study. The subjects were seated comfortably in armchairs. The probe was fastened symmetrically to the forehead of subjects, as previously described. The optical density at two wavelengths (740 nm and 850 nm) and as well as the EEG signals were sampled at a rate of 512 samples per second. Conductive paste (Ten20, Weaver) was applied to the EEG Ag/AgCl electrode on the probe, in order to minimize the skin-electrode impedance. The experiment was conducted at room temperature (22-24° C.) and with under normal ambient light. The relative changes of HHb, HbO₂ and total Hb were measured as hemodynamic variation parameters. The EEG signals were recorded concurrently.

The subject was asked to breathe through the mask until arterial oxygenation as measured by the pulse oximeter approached the 85%-95% blood oxygen saturation. Afterward, the subject was instructed to remove the mask and asked to breathe normally for two minutes.

The hemodynamic changes in the brain, resulting from hypoxia, were detected by the NIRS recording apparatus and were observed to occur earlier than changes in peripheral oxygenation. This delay varied from 10 to 15 seconds among subjects.

CO₂-dependant low-frequency EEG potential changes have been studied previously and were found to be mostly associated with the activity of apical dendrites of cortical neurons. In the following study, NIRS hemodynamic estimation was used as a supplementary parameter for recording the neurophysiological activity of the brain.

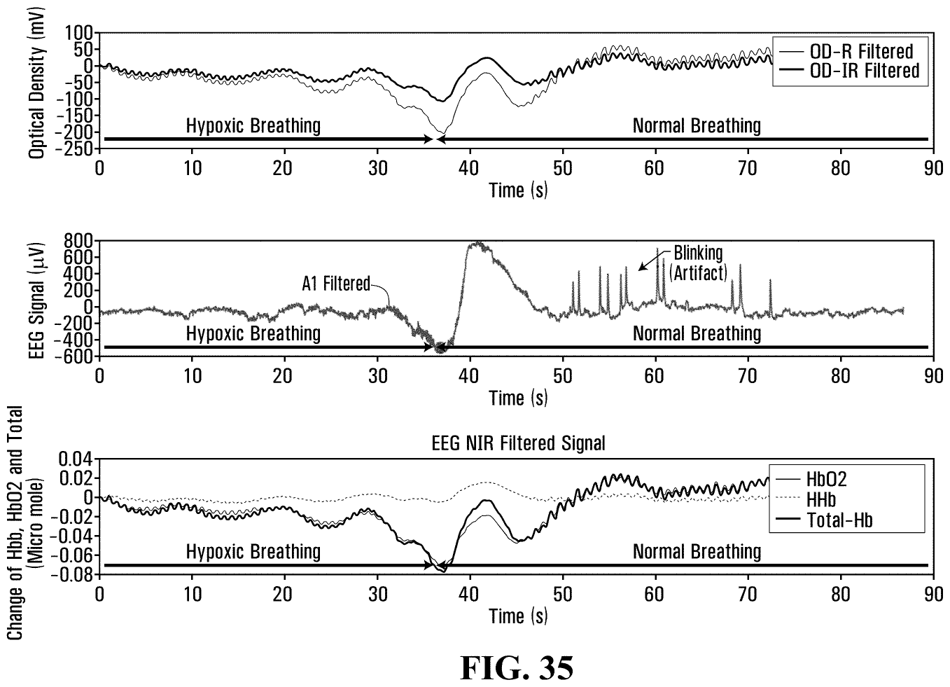

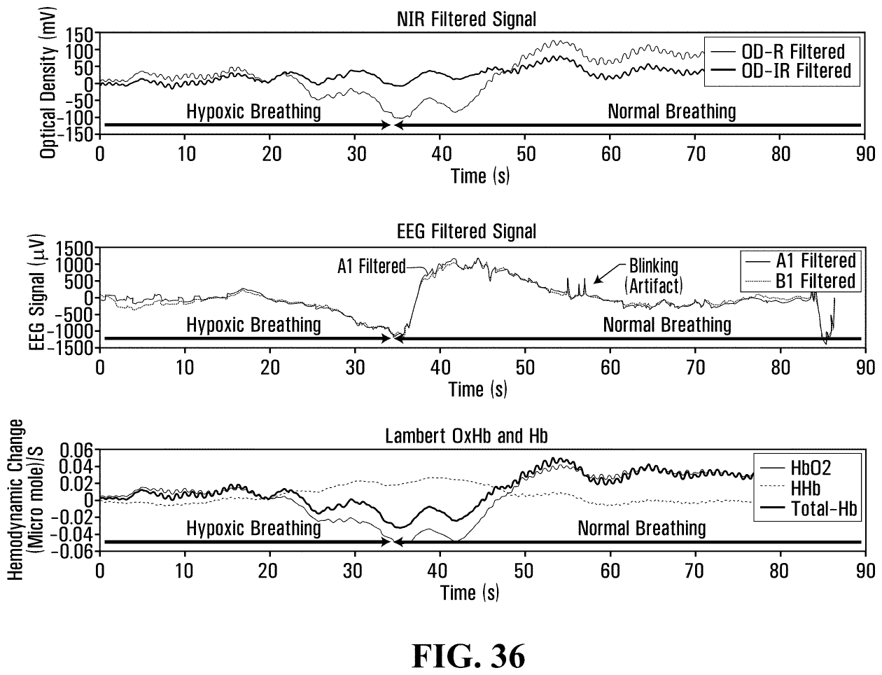

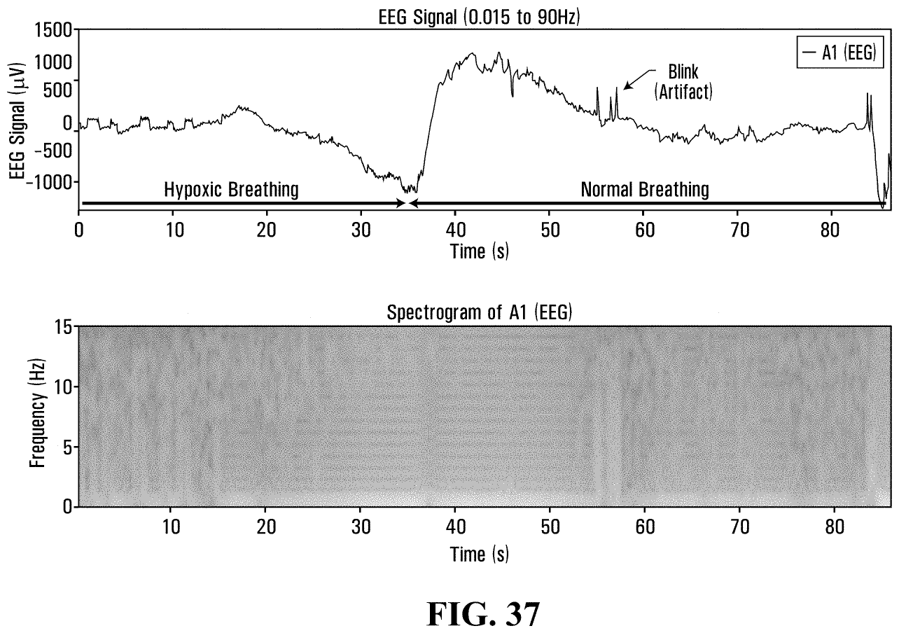

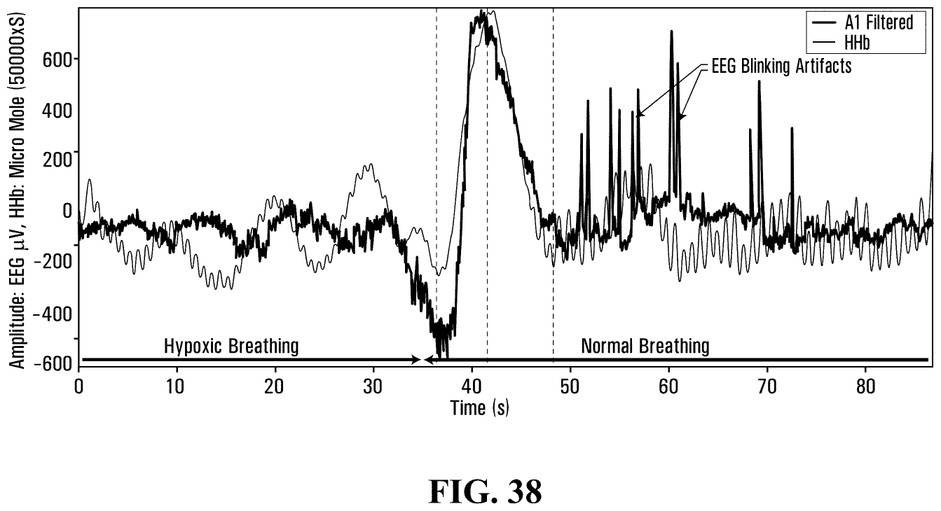

It was proposed to measure the full-band EEG and concurrent hemodynamic changes during hypoxic breathing conditions. Non-invasive surface electrodes were used. The NIRS optodes were positioned on the forehead to monitor the frontal lobe; the Ag/AgCl electrodes were interspersed between the NIRS optodes. Two EEG electrodes monitored electric potentials in the sensory cortex. The pulse oximetry device on the left finger verified the effectiveness of the altitude simulation maneuver, as well as allowed a physician to continually monitor the safety and well-being of the subjects. The hypoxic breathing conditions led to variation in CO₂ concentration (or partial pressure of CO₂: pCO₂). In all five subjects, the EEG measured from the scalp displayed low-frequency shifts during hypocapnia. As shown FIG. 35, the EEG low-frequency shifts appeared after the observed hemodynamic changes. As shown in FIG. 36, the graph of hemodynamic response and concurrent EEG illustrates two relevant low-frequency EEG signals, recorded at the forehead (A1 left side) and the sensory cortex (10/10 EEG electrode system, CP3(16)). The spectrum analysis of the EEG signal, using STFT, is illustrated in FIG. 37 and shows the low-frequency activity of the EEG signal. The superposition of HHb and the EEG signal, recorded at the frontal lobe on the left side, is illustrated in FIG. 38. The EEG changes induced by CO₂ concentration are proportional to the HHb parameter recorded by the NIRS subsystem.

The association between HHb and slow EEG signals as observed in this study was analyzed. The results of this analysis included slow-EEG signals and the concurrent change of CO₂ concentration. The aforementioned results appear to be in agreement with those of previously reported studies. Consequently, the EEG shifts, measured on the surface of the scalp, were non-neuronal EEG and did not represent the low-frequency activity of the neurons.

While the disclosure has been described in connection with specific embodiments, it is to be understood that the disclosure is not limited to these embodiments, and that alterations, modifications, and variations of these embodiments may be carried out by the skilled person without departing from the scope of the disclosure. In particular, while the disclosure refers to specific makes and models of components, as the skilled person would recognize the disclosure is not limited to such makes and models of components. Rather, the disclosure extends to any suitable component that falls within the scope of the claims that follow. It is furthermore contemplated that any part of any aspect or embodiment discussed in this specification can be implemented or combined with any part of any other aspect or embodiment discussed in this specification.

1. An electroencephalography (EEG) device comprising:
one or more inputs for connecting via electrodes to a subject;
one or more DC/AC EEG amplifiers for amplifying electric potential signals from a subject, wherein each DC/AC EEG amplifier comprises:
a preamplifier connected to the one or more inputs and configured to amplify a signal received at the one or more inputs, the preamplifier having a differential gain set so as to have a common mode rejection ratio (CMRR) of at least 100 dB; and
an amplifier circuit connected to the preamplifier and configured to further amplify the signal and, based thereon, output an output signal comprising a DC component having a frequency of less than 0.02 Hz.

2. The EEG device of claim 1, wherein the one or more inputs comprise multiple inputs including at least one reference input and one or more EEG inputs, and wherein the EEG device further comprises a reference circuit connected to the at least one reference input and configured to output a reference signal based on a signal received at the at least one reference input.

3. The EEG device of claim 2, wherein the preamplifier is further connected to the reference circuit and is further configured to amplify the signal received at the one or more EEG inputs.

4. (canceled)

5. The EEG device of claim 1, wherein the CMRR is at least 100 dB at 50 Hz or 60 Hz.

6. The EEG device of claim 1, wherein the DC component has a frequency of from 0.015 Hz to 0.02 Hz.

7. The EEG device of claim 1, wherein the output signal further comprises an AC component having a frequency of from 0.02 Hz to 80 Hz.

8. (canceled)

9. (canceled)

10. (canceled)

11. The EEG device of claim 1, wherein the differential gain is set to less than 200.

12. (canceled)

13. The EEG device of claim 1, wherein each preamplifier comprises an operational amplifier having an input impedance greater than 10 GΩ.

14. The EEG device of claim 1, wherein each preamplifier comprises an operational amplifier having an input bias current of less than 10 pA.

15. The EEG device of claim 2, wherein the reference signal is buffered prior to being received at the preamplifier.

16. The EEG device of claim 1, further comprising a Driven Right Leg (DRL) circuit for connecting via one or more electrodes to a subject, and for reducing a common mode voltage.

17. The EEG device of claim 1, wherein the differential gain is set by a single resistive component.

18. The EEG device of claim 2, wherein the reference circuit comprises a multi-turn potentiometer for compensating resistor tolerance.

19. The EEG device of claim 1, wherein each preamplifier does not comprise a high-pass filter.

20. The EEG device of claim 2, wherein the reference circuit comprises a transient voltage suppression (TVS) diode, and wherein each preamplifier comprises a TVS diode connected at an input thereto.

21. The EEG device of claim 1, wherein a first multilayer PCB comprises each preamplifier in a topmost layer of the first PCB, and the one or more inputs in a bottommost layer of the first PCB.

22. (canceled)

23. (canceled)

24. The EEG device of claim 1, wherein the DC component has a frequency indicative of cortical spreading depression.

25. The EEG device of claim 1, wherein each amplifier circuit:
does not comprise a high-pass filter; or
comprises an integrator.

26. A method of recording electroencephalography (EEG) signals from a subject, comprising: attaching one or more electrodes to the subject's scalp, the electrodes connected to one or more inputs of an EEG device, the EEG device comprising one or more DC/AC EEG amplifiers for amplifying electric potential signals from the subject, wherein each DC/AC EEG amplifier comprises:
a preamplifier connected to the one or more inputs and configured to amplify a signal received at the one or more inputs, the preamplifier having a differential gain set so as to have a common mode rejection ratio (CMRR) of at least 100 dB; and
an amplifier circuit connected to the preamplifier and configured to further amplify the signal and, based thereon, output an output signal comprising a DC component having a frequency of less than 0.02 Hz; and
using the EEG device to record electric potentials of the subject.

27. The method of claim 26, wherein the subject is epileptic.

28.-45. (canceled)
